(19) United States
(12) Patent Application Publication
(10) Pub. No.: US 2016/0017424 A1
(43) Pub. Date: Jan. 21, 2016

(54) COLLECTION OF PROBES FOR AUTISTIC SPECTRUM DISORDERS AND THEIR USE

(71) Applicants: THE RESEARCH FOUNDATION OF STATE UNIVERSITY OF NEW YORK, Syracuse, NY (US); THE SCRIPPS RESEARCH INSTITUTE, La Jolla, CA (US); THE REGENTS OF THE UNIVERSITY OF CALIFORNIA, Oakland, CA (US)

(72) Inventors: Stephen GLATT, Syracuse, NY (US); Ming TSUANG, La Jolla, CA (US); Eric COURCHESNE, La Jolla, CA (US); Nicholas J. SCHORK, La Jolla, CA (US)

(21) Appl. No.: 14/770,205

(22) PCT Filed: Feb. 14, 2014
(86) PCT No.: PCT/US2014/016537

§ 371 Date: Aug. 25, 2015

Related U.S. Application Data
(60) Provisional application No. 61/769,000, filed on Feb. 25, 2013.

(51) Int. Cl. C12Q 1/68

(52) U.S. Cl. CPC C12Q 1/6883; C12Q 2600/158

(57) ABSTRACT

The present invention relates to collections of probes or their complements that recognize a set of biomarkers related to autism spectrum disorders (ASDs), as well as to methods utilizing these probes for diagnosing whether a subject has an ASD or has a predisposition for developing an ASD.

This application claims the priority benefit of U.S. Provisional Patent Application Ser. No. 61/769,000, filed Feb. 25, 2013, which is hereby incorporated by reference in its entirety.

This invention was made with government support under United States National Institutes of Health Grant No. 5P50MH081755-003. The government has certain rights in this invention.

### FIELD OF THE INVENTION

The present invention is directed to collections of probes for autistic disorders and their use.

### BACKGROUND OF THE INVENTION

Autism spectrum disorders (ASDs) include Autistic Disorder (autism), Asperger Disorder, and Pervasive Developmental Disorder—Not Otherwise Specified (PDD-NOS). PDD-NOS is characterized by developmental delays of sociability, communication and use imagination. Asperger's syndrome is a more severe form of PDD-NOS but lacks the language and intelligence deficits normally associated with autism. Autism is exemplified by severe communication impairments, social interaction deficits and repetitive stereotypic behaviors. Each of these disorders has specific diagnostic criteria as outlined by the American Psychiatric Association (APA) in its Diagnostic & Statistical Manual of Mental Disorders (DSM-IV-TR).

In the first description of the disease, Kanner suggested an influence of child-rearing practices on the development of autism, after observing similar traits in parents of the affected children. While experimental data fail to support several environmental hypotheses, there has been growing evidence for a strong genetic influence on this disorder. The rate of autism in siblings of affected individuals was shown to be a 2-6%, two orders of magnitude higher than in the general population. Twin studies have demonstrated significant differences in monozygotic and dizygotic twin concordance rates, the former concordant in 60% of twin pairs, with most of the non-autistic monozygotic co-twins displaying milder related social and communicative abnormalities. Social, language, and cognitive difficulties have also been found among relatives of autistic individuals in comparison to the relatives of controls. The heritability of autism has been estimated to be >90%.

Prevalence estimates for ASDs have been reported to be approximately 1 in every 100 children in the general population. In families with an autistic child, recurrence rates are estimated to be greater than 15% that an additional offspring will also have autism (Landa et al., “Social and Communication Development in Toddlers with Early and Later Diagnosis of Autism Spectrum Disorders,” Arch Gen Psychiatry 64:853-64 (2007); Landa R J, “Diagnosis of Autism Spectrum Disorders in the First 3 Years of Life,” Nat Clin Pract Neurol 138-47 (2008)). These disorders are among the most severe and debilitating disorders of early brain development, typically emerging between infancy and early childhood, and commonly imposing lifelong disability on affected children as well as considerable strain on their caretakers and society at large.

The current state-of-the-art diagnosis of ASD is a series of various behavioral questionnaires. A brief observation in a single setting cannot present a true picture of an individual's abilities and behaviors. Parental and other caregivers' input and developmental history are very important components of making an accurate diagnosis. At first glance, some persons with autism may appear to have mental retardation, a behavior disorder, problems with hearing, or even odd and eccentric behavior. To complicate matters further, these conditions can co-occur with autism. Because the ASD phenotype is so complicated, a molecular-based test would greatly improve the accuracy of diagnosis at an earlier age, when phenotypic/behavioral assessment is not possible, or integrated with phenotypic/behavioral assessment. Also, diagnosis at an earlier age would allow initiation of ASD treatment at an earlier age which may be beneficial to short and long-term outcomes.

In the last decade, autism diagnoses have increased by 300% to 500% in the United States and many other countries. Accordingly there is a need to diagnose ASD in subjects suspected of having ASD or to identify subjects that are at risk of developing an ASD. One way to combat ASDs would be to discover biological signatures—or “biomarkers”—of early risk for ASDs that could be exploited for early detection and treatment and could thereby improve long-term clinical outcomes.

The current standard for diagnosing ASDs solely involves behavioral analysis. There are currently no biomarkers or medical tests available, and, as a result, diagnosis is delayed until 1.5 to 4 years of age. Most prior transcriptome-wide studies of gene expression in ASDs have used post mortem brain tissue or transformed lymphoblastoid cell lines (Purcell et al., “Postmortem Brain Abnormalities of the Glutamate Neurotransmitter System in Autism,” Neurology 57:1618-1628 (2001); Chow et al., “Age-Dependent Brain Gene Expression and Copy Number Anomalies in Autism Suggest Distinct Pathological Processes at Young Versus Mature Ages,” PLoS Genet 8:e1002592 (2012)). Both approaches are quite useful and revealing, often observing dysregulation of viable candidate pathways in autism, but both have limitations, such as the inability to fully account for agonal factors or the effects of viral transformation on the observed gene expression differences in autism. In contrast, Kuwano et al. sought autism biomarkers in peripheral leukocytes of young adults with autism, observing dysregulation of 16 genes related to cell morphology, cellular assembly and organization, and nerve system development and function (Kuwano et al., “Autism-Associated Gene Expression in Peripheral Leucocytes Commonly Observed Between Subjects With Austism and Healthy Women Having Autistic Children,” PLoS One 6:e24723 (2001)). Albeit informative regarding persistent gene expression abnormalities in peripheral blood in individuals with ASDs, this study cannot shed light on the peripheral blood transcriptomic events accompanying the emergence of autism in its earliest developmental stages of infancy or toddlerhood.

The present invention is directed to overcoming these and other limitations in the art.

### SUMMARY OF THE INVENTION

A first aspect of the present invention relates to a collection of probes or their complements that recognize biomarkers comprising at least 50% of the biomarkers from one of the following biomarker sets: (1) ZNF329, LOC641518, TAP1, GBP2, RAB3IP, and MYOF biomarkers; (2) MRPS10, ARF3, CLORF85, KCNE1L, BIN2, CACHD1, CYB5R3, FKBP12-EXI, CHM, DUS4L, STX5, AK3, BU580973, TCRA, CR608770, and SPIT biomarkers; (3) SC65, FUNDC2, NDRG2, RPL28, SRP54, LOC643466, ZDHHC11B, NSUN5B, NDRG3, DHRS3, CPEB2, RAB3IP, PPID, FOXP1, and EFNA1 biomarkers; (4) C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, and MAP4K5 biomarkers; (5) CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, and NSUN5 biomarkers; (6) GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, and EFNA1 biomarkers; or (7) GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1 biomarkers.

The present invention is also directed to a method of diagnosing whether a subject has an ASD that involves obtaining a biological sample from a subject potentially having an ASD and providing a collection of probes recognizing biomarkers comprising as least 50% of the biomarkers from one of the following biomarker sets: (1) ZNF329, LOC641518, TAP1, GBP2, RAB3IP, and MYOF; (2) MRPS10, ARF3, CLORF85, KCNE1L, BIN2, CACHD1, CYB5R3, FKBP12-EXI, CHM, DUS4L, STX5, AK3, BU580973, TCRA, CR608770, and SPI1; (3) SC65, FUNDC2, NDRG2, RPL28, SRP54, LOC643466, ZDHHC11B, NSUN5B, NDRG3, DHRS3, CPEB2, RAB3IP, PPID, FOXP1, and EFNA1; or (4) C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, and MAP4K5. The biological sample is then contacted with said collection of probes under conditions effective to permit hybridization of said probes to complementary nucleic acid molecules, if present, in the sample. This method further involves detecting any hybridization as a result of said contacting and identifying whether the subject has an ASD based on said detecting.

Another aspect of the present invention is directed to a method of determining whether a subject has a predisposition for developing an ASD. This method involves obtaining a biological sample from a subject at risk of potentially having a predisposition for developing an ASD and providing a collection of probes recognizing biomarkers comprising at least 50% of the biomarkers from one of the following biomarker sets: (1) CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, and NSUN5; (2) GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, and EFNA1; or (3) GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1. The biological sample from the subject is then contacted with said collection of probes under conditions effective to permit hybridization of said probes to complementary nucleic acid molecules, if present, in the sample. This method further involves detecting any hybridization as a result of said contacting, and identifying whether or not the subject has a predisposition for developing an ASD based on said detecting.

The present invention is also directed to a method of diagnosing whether a subject has an autism spectrum disorder involving obtaining a biological sample from a subject potentially having an autism spectrum disorder, providing one or more probes recognizing at least 50% of the following biomarkers: ZNF329, LOC641518, TAP1, GBP2, RAB3IP, MYOF, MRPS10, ARF3, CLORF85, KCNE1L, BIN2, CACHD1, CYB5R3, FKBP12-EXI, CHM, DUS4L, STX5, AK3, BU580973, TCRA, CR608770, SPI1, SC65, FUNDC2, NDRG2, RPL28, SRP54, LOC643466, ZDHHC11B, NSUN5B, NDRG3, DHRS3, CPEB2, RAB3IP, PPID, FOXP1, EFNA1, C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, MAP4K5, CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, NSUN5, GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, EFNA1, GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1 biomarkers, contacting the biological sample from the subject with said collection of probes under conditions effective to permit hybridization of said probes to complementary nucleic acid molecules, if present, in the sample, detecting any hybridization as a result of said contacting, and identifying whether the subject has an autism spectrum disorder based on said detecting.

A final aspect of the present invention relates to a method of diagnosing whether a subject has a predisposition for developing an autism spectrum disorder involving obtaining a biological sample from a subject potentially having a predisposition for developing an autism spectrum disorder, providing one or more probes recognizing at least 50% of the following biomarkers: ZNF329, LOC641518, TAP1, GBP2, RAB3IP, MYOF, MRPS10, ARF3, CLORF85, KCNE1L, BIN2, CACHD1, CYB5R3, FKBP12-EXI, CHM, DUS4L, STX5, AK3, BU580973, TCRA, CR608770, SPI1, SC65, FUNDC2, NDRG2, RPL28, SRP54, LOC643466, ZDHHC11B, NSUN5B, NDRG3, DHRS3, CPEB2, RAB3IP, PPID, FOXP1, EFNA1, C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, MAP4K5, CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, NSUN5, GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, EFNA1, GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1 biomarkers, contacting the biological sample from the subject with said collection of probes under conditions effective to permit hybridization of said probes to complementary nucleic acid molecules, if present, in the sample, detecting any hybridization as a result of said contacting, and identifying whether the subject has a predisposition for developing an autism spectrum disorder based on said detecting.

The present invention provides reliable diagnostics to more people, facilitate primary and differential diagnoses, and, importantly, result in earlier identification of the disorder in affected children. This will lead to earlier intervention and a more favorable prognosis. The identification of ASD-specific biomarkers could revolutionize the diagnosis and treatment of these disorders. For example, the availability of ASD biomarkers could expedite and standardize the diagnostic process, which presently involves considerable time, effort, and uncertainty. By allowing earlier identification, biomarkers could hasten the provision of effective treatment and improve prognoses. Further, biomarkers could form the basis for prevention efforts targeting at-risk individuals, which could reduce the morbidity and prevalence of these conditions. As ASDs are very heterogeneous conditions, biomarker research may also help differentiate subtypes of ASDs, which in turn may shed light on the distinct etiologies of these conditions. Collectively, these advances would translate into an enormous improvement in global public health and substantially decrease the suffering of affected individuals and families.

### DETAILED DESCRIPTION OF THE INVENTION

A first aspect of the present invention relates to a collection of probes or their complements that recognize biomarkers comprising at least 50% of the biomarkers from one of the following biomarker sets: (1) ZNF329, LOC641518, TAP1, GBP2, RAB3IP, and MYOF biomarkers; (2) MRPS10, ARF3, CLORF85, KCNE1L, BIN2, CACHD1, CYB5R3, FKBP12-EXI, CHM, DUS4L, STX5, AK3, BU580973, TCRA, CR608770, and SPIT biomarkers; (3) SC65, FUNDC2, NDRG2, RPL28, SRP54, LOC643466, ZDHHC11B, NSUN5B, NDRG3, DHRS3, CPEB2, RAB3IP, PPID, FOXP1, and EFNA1 biomarkers; (4) C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, and MAP4K5 biomarkers; (5) CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, and NSUN5 biomarkers; (6) GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, and EFNA1 biomarkers; or (7) GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1 biomarkers.

ZNF329 has the nucleotide sequence corresponding to the NCBI Reference Sequence: NM_{—}024620.3, which is hereby incorporated by reference in its entirety. An example of a probe that hybridizes with ZNF329 has the nucleotide sequence of SEQ ID NO: 1 as shown below:

CCCCACCACTACCTCCATGGTTGTTCATTTAGGATGCTTCTAATTCAGCC

LOC641518 has the nucleotide sequence corresponding to the NCBI Reference Sequence: XR_{—}017788.1, which is hereby incorporated by reference in its entirety. An example of a probe that hybridizes with LOC641518 has the nucleotide sequence of SEQ ID NO: 2 as shown below:

GGGGCTATATTCTCAGGCTTCCTGGGCTCTGTTGGCATTGGGCAAGCACG

TAP1 has the nucleotide sequence corresponding to the NCBI Reference Sequence: NM_{—}000593.5, which is hereby incorporated by reference in its entirety. An example of a probe that hybridizes with TAP1 has the nucleotide sequence of SEQ ID NO: 3 as shown below:

GTAACGGAGTTTAGAGCCAGGGCTGATGCTTTGGTGTGGCCAGCACTCTG

GBP2 has the nucleotide sequence corresponding to the NCBI Reference Sequence: NM_{—}004120.3, which is hereby incorporated by reference in its entirety. An example of a probe that hybridizes with GBP2 has the nucleotide sequence of SEQ ID NO: 4 as shown below:

GCCTGTCCAGCTCCCTCTCCCCAAGAAACAACATGAATGAGCAACTTCAG

RAB3IP has the nucleotide sequence corresponding to the NCBI Reference Sequence: NM_{—}175624.2, which is hereby incorporated by reference in its entirety. An example of a probe that hybridizes with RAB3IP has the nucleotide sequence of SEQ ID NO: 5 as shown below:

GGAACTCTGATGCTCTGCGTGGGACCATGCCTGAACTCCCCGAATAACTG

MYOF has the nucleotide sequence corresponding to the NCBI Reference Sequence: NM_{—}133337.1, which is hereby incorporated by reference in its entirety. An example of a probe that hybridizes with MYOF has the nucleotide sequence of SEQ ID NO: 6 as shown below:

GCCATGTCACCGAGCCCCATTGATTCCCAGAGGGTCTTAGTCCTGGAAAG

MRPS10 has the nucleotide sequence corresponding to the NCBI Reference Sequence: NM_{—}018141.2, which is hereby incorporated by reference in its entirety. An example of a probe that hybridizes with MRPS10 has the nucleotide sequence of SEQ ID NO: 7 as shown below:

GTAGAAGCAGGTCCATGTCTTTTGTGGTTTCCTGCACATCTTTGGAGTAG

ARF3 has the nucleotide sequence corresponding to NCBI Reference Sequence: NM_{—}001659.1, which is hereby incorporated by reference in its entirety. An example of a probe that hybridizes with ARF3 has the nucleotide sequence of SEQ ID NO: 8 as shown below:

AGGGACCAATCTGGGGCTGGAAATGTTAGGAGGTTGCCTTGGTGCTGCCC

C1ORF85 has the nucleotide sequence corresponding to NCBI Reference Sequence: NM_{—}144580.1, which is hereby incorporated by reference in its entirety. An example of a probe that hybridizes with C1ORF85 has the nucleotide sequence of SEQ ID NO: 9 as shown below:

GGGACAGGGCTATTGATAAGGTCCCCTTGGTGTTGCCTTCTTGCATCTCC

KCNE1L has the nucleotide sequence corresponding to NCBI Reference Sequence: NM_{—}012282.2, which is hereby incorporated by reference in its entirety. An example of a probe that hybridizes with KCNE1L has the nucleotide sequence of SEQ ID NO: 10 as shown below:

TCTTGCCTCCCCAGTTTTCATCCCAGTGGTAACGCCTGATTTTTGGTAGC

BIN2 has the nucleotide sequence corresponding to NCBI Reference Sequence: NM_{—}016293.2, which is hereby incorporated by reference in its entirety. An example of a probe that hybridizes with BIN2 has the nucleotide sequence of SEQ ID NO: 11 as shown below:

GGTTGCTTCAGAGCCTGGAGAGGCAAAGAAGATGGAAGACAAGGAAAAGG

CACHD1 has the nucleotide sequence corresponding to NCBI Reference Sequence: NM_{—}020925.2, which is hereby incorporated by reference in its entirety. An example of a probe that hybridizes with CACHD1 has the nucleotide sequence of SEQ ID NO: 12 as shown below:

CACGGTTAAAAGCTGCTGCCAGTTAGCCAAGACATTATCCACCAAATTGC

CYB5R3 has the nucleotide sequence corresponding to NCBI Reference Sequence: NM_{—}000398.4, which is hereby incorporated by reference in its entirety. An example of a probe that hybridizes with CYB5R3 has the nucleotide sequence of SEQ ID NO: 13 as shown below:

GGGCCCTCCCAGAACCTCAGCATTTCCTTCCAGCCCATCCAAACACTGAG

FKBP12-EXI has the nucleotide sequence corresponding to NCBI Reference Sequence: XM_{—}925989.1, which is hereby incorporated by reference in its entirety. An example of a probe that hybridizes with FKBP12-EXI has the nucleotide sequence of SEQ ID NO: 14 as shown below:

TCCCTGGGCCAGCAGGGACCTCTGAAGCCTTCTTTGTGGCCTTATTTTTT

CHM has the nucleotide sequence corresponding to NCBI Reference Sequence: NM_{—}000390.2, which is hereby incorporated by reference in its entirety. An example of a probe that hybridizes with CHM has the nucleotide sequence of SEQ ID NO: 15 as shown below:

CTGCCATAGTTACCTGGATTGTCAGCCTTGGTAGCCTTTGTCTAAAGTCC

DUS4L has the nucleotide sequence corresponding to NCBI Reference Sequence: NM_{—}181581.1, which is hereby incorporated by reference in its entirety. An example of a probe that hybridizes with DUS4L has the nucleotide sequence of SEQ ID NO: 16 as shown below:

GTGCCTCTCAGCCGTTGATTGTAACTTTAAAGTCCCATGGTTTTGGAGTG

STX5 has the nucleotide sequence corresponding to NCBI Reference Sequence: NM_{—}003164.3, which is hereby incorporated by reference in its entirety. An example of a probe that hybridizes with STX5 has the nucleotide sequence of SEQ ID NO: 17 as shown below:

GGAACAGGAGGAAACCATTCAGAGGATCGACGAGAACGTGCTAGGAGCCC

AK3 has the nucleotide sequence corresponding to NCBI Reference Sequence: NM_{—}016282.2, which is hereby incorporated by reference in its entirety. An example of a probe that hybridizes with AK3 has the nucleotide sequence of SEQ ID NO: 18 as shown below:

GTGGCTATTACTTTGTTCTTGGTCCTTCACAGGGCCTGCTCCATCCCACC

BU580973 has the nucleotide sequence corresponding to NCBI Reference Sequence: BU580973, which is hereby incorporated by reference in its entirety. An example of a probe that hybridizes with BU580973 has the nucleotide sequence of SEQ ID NO: 19 as shown below:

GCCTGTTGCTTTGGCTTGTATTTTCAGTGCTGTGTTGAATAAGAGTGGTG

TCRA has the nucleotide sequence corresponding to NCBI Reference Sequence: AY375451, which is hereby incorporated by reference in its entirety. An example of a probe that hybridizes with TCRA has the nucleotide sequence of SEQ ID NO: 20 as shown below:

GCGCGAAGAGCAACTATCAGTTAATCTGGGGCGCTGGGACCAAGCTAATT

CR608770 has the nucleotide sequence corresponding to NCBI Reference Sequence: AL528570, which is hereby incorporated by reference in its entirety. An example of a probe that hybridizes with CR608770 has the nucleotide sequence of SEQ ID NO: 21 as shown below:

TGCACACTGCCCTATTGTAGTCAAGTCTTACCCCCACCTCACATCTCCTG

SPI1 has the nucleotide sequence corresponding to NCBI Reference Sequence: NM_{—}003120.2, which is hereby incorporated by reference in its entirety. An example of a probe that hybridizes with SPI1 has the nucleotide sequence of SEQ ID NO: 22 as shown below:

ACGCCAGCTGGGCGTCAGACCCCACCGGGGCAACCTTGCAGAGGACGACC

SC65 has the nucleotide sequence corresponding to NCBI Reference Sequence: NM_{—}006455.2, which is hereby incorporated by reference in its entirety. An example of a probe that hybridizes with SC65 has the nucleotide sequence of SEQ ID NO: 23 as shown below:

TCTGGACCACAGGATGGTGGTGGCATTGCAGGTTGGCAAGTGGGCTGATG

FUNDC2 has the nucleotide sequence corresponding to NCBI Reference Sequence: NM_{—}023934.3, which is hereby incorporated by reference in its entirety. An example of a probe that hybridizes with FUNDC2 has the nucleotide sequence of SEQ ID NO: 24 as shown below:

CCTGCTTCCCCACACTCTGCCTCTCCTGACATCTGGGTCTCTGGGTTATG

NDRG2 has the nucleotide sequence corresponding to NCBI Reference Sequence: NM_{—}201539.1, which is hereby incorporated by reference in its entirety. An example of a probe that hybridizes with NDRG2 has the nucleotide sequence of SEQ ID NO: 25 as shown below:

GTTGGAATGGGAGTTGGCGGGCAGTGAACGAGTGTGGGGAAGGATTGGTG

RPL28 has the nucleotide sequence corresponding to NCBI Reference Sequence: NM_{—}000991.3, which is hereby incorporated by reference in its entirety. An example of a probe that hybridizes with RPL28 has the nucleotide sequence of SEQ ID NO: 26 as shown below:

CCCAAGCACCTGGAAGACATGCCAGATCCATGTGCAGTAATGCCTGGTGG

SRP54 has the nucleotide sequence corresponding to NCBI Reference Sequence: XM_{—}939727.1, which is hereby incorporated by reference in its entirety. An example of a probe that hybridizes with SRP54 has the nucleotide sequence of SEQ ID NO: 27 as shown below:

GTTATATAGCTATACAGAAATGGATCCTGTCATCATTGCTTCTGAAGGAG

LOC643466 has the nucleotide sequence corresponding to NCBI Reference Sequence: XM_{—}926789.1, which is hereby incorporated by reference in its entirety. An example of a probe that hybridizes with LOC643466 has the nucleotide sequence of SEQ ID NO: 28 as shown below:

TTGACTCAAACCCACGGTGCGCCTCGGGCCGTTAGGGGTACCCCGAGGCA

ZDHHC11B has the nucleotide sequence corresponding to NCBI Reference Sequence: XM_{—}942335.1, which is hereby incorporated by reference in its entirety. An example of a probe that hybridizes with ZDHHC11B has the nucleotide sequence of SEQ ID NO: 29 as shown below:

TGGGGCATGTGGGCTCTGCACTACCTCCAGCTGACCATGGCATTCAGTTG

NSUN5B has the nucleotide sequence corresponding to NCBI Reference Sequence: NM_{—}001039575.1, which is hereby incorporated by reference in its entirety. An example of a probe that hybridizes with NSUN5B has the nucleotide sequence of SEQ ID NO: 30 as shown below:

CGGGGCGGGGAAGTGAACCCCGACGGTCAGCGCTTTGTCATCTGGTTTCA

NDRG3 has the nucleotide sequence corresponding to NCBI Reference Sequence: NM_{—}022477.2, which is hereby incorporated by reference in its entirety. An example of a probe that hybridizes with NDRG3 has the nucleotide sequence of SEQ ID NO: 31 as shown below:

CTACTGTTAGGTGAGGGAGTCACAGCCAGACAGAGAGTATTGCTGGAGGG

DHRS3 has the nucleotide sequence corresponding to NCBI Reference Sequence: NM_{—}004753.4, which is hereby incorporated by reference in its entirety. An example of a probe that hybridizes with DHRS3 has the nucleotide sequence of SEQ ID NO: 32 as shown below:

TGACCCCCACAGGGAGGCAGGAAAACAGCCAGAAGCCACCTTGACACTTT

CPEB2 has the nucleotide sequence corresponding to NCBI Reference Sequence: NM_{—}182646.1, which is hereby incorporated by reference in its entirety. An example of a probe that hybridizes with CPEB2 has the nucleotide sequence of SEQ ID NO: 33 as shown below:

TCAGAGCAGTGGCTGGGGCACTGGAAGTATGTCCTGGGGAGCAATGCATG

PPID has the nucleotide sequence corresponding to NCBI Reference Sequence: NM_{—}005038.2, which is hereby incorporated by reference in its entirety. An example of a probe that hybridizes with PPID has the nucleotide sequence of SEQ ID NO: 34 as shown below:

CAGGTGTGAGCCACCGTGCCCGGCCAAGTAAAATGTTTTTTAAAATGGTT

FOXP1 has the nucleotide sequence corresponding to NCBI Reference Sequence: NM_{—}032682.5, which is hereby incorporated by reference in its entirety. An example of a probe that hybridizes with FOXP1 has the nucleotide sequence of SEQ ID NO: 35 as shown below:

AGAATATTGGATGACATTTCCTGACATGTGGGAGGGAGAAACTCCCTAAC

EFNA1 has the nucleotide sequence corresponding to NCBI Reference Sequence: NM_{—}004428.2, which is hereby incorporated by reference in its entirety. An example of a probe that hybridizes with EFNA1 has the nucleotide sequence of SEQ ID NO: 36 as shown below:

CCTGCCTTTAAGCCAAAGAAACAAGCTGTGCAGGCATGGTCCCTTAAG
GC.

An example of another probe that hybridizes with EFNA1 has the nucleotide sequence of SEQ ID NO: 37 as shown below:

CAGGGCCCACGTGTATAGTATCTGTATATAAGTTGCTGTGTGTCTGTCCT

C5ORF44 has the nucleotide sequence corresponding to NCBI Reference Sequence: NR_{—}003545.1, which is hereby incorporated by reference in its entirety. An example of a probe that hybridizes with C5ORF44 has the nucleotide sequence of SEQ ID NO: 38 as shown below:

TGTGGGTCAAAAGTGCCGGTCAAAATGGAAGTGAATCCCCCTAAACAGGA

ARHGAP25 has the nucleotide sequence corresponding to NCBI Reference Sequence: NM_{—}001007231.1, which is hereby incorporated by reference in its entirety. An example of a probe that hybridizes with ARHGAP25 has the nucleotide sequence of SEQ ID NO: 39 as shown below:

GGGAAAGAGTGAAAAGACAAGAAGGGCGCAAACTGTGACAGACTCACCGC

CTDSPL2 has the nucleotide sequence corresponding to NCBI Reference Sequence: NM_{—}016396.2, which is hereby incorporated by reference in its entirety. An example of a probe that hybridizes with CTDSPL2 has the nucleotide sequence of SEQ ID NO: 40 as shown below:

CCTGTGGCGCAGTACACTCCCAAGCCACCAATGCAGTTAATATGCTCTCA

CKAP2 has the nucleotide sequence corresponding to NCBI Reference Sequence: NM_{—}001098525.1, which is hereby incorporated by reference in its entirety. An example of a probe that hybridizes with CKAP2 has the nucleotide sequence of SEQ ID NO: 41 as shown below:

CTTGCGTCCCTTGGACTGCCTGTTGATTGATGGAAAGTGTCTGCACTGAC

MAZ has the nucleotide sequence corresponding to NCBI Reference Sequence: NM_{—}001042539.1, which is hereby incorporated by reference in its entirety. An example of a probe that hybridizes with MAZ has the nucleotide sequence of SEQ ID NO: 42 as shown below:

GTTGGTTGCGGGGGAGAGGGGAGAATGGAGTAGAGTCCCTTGGTACAAGC

BET1 has the nucleotide sequence corresponding to NCBI Reference Sequence: NM_{—}005868.4, which is hereby incorporated by reference in its entirety. An example of a probe that hybridizes with BET1 has the nucleotide sequence of SEQ ID NO: 43 as shown below:

GGAAACTTAGTGGGAGAGTAACAGAATGCCTGGAGAGCCTGACTCTGAGC

CR617556 has the nucleotide sequence corresponding to NCBI Reference Sequence: XR_{—}015150.1, which is hereby incorporated by reference in its entirety. An example of a probe that hybridizes with CR617556 has the nucleotide sequence of SEQ ID NO: 44 as shown below:

CAGCATAGACTTAACTCCCTTAAGCCCAGACATCTGTTGGGACCTGACCC

RPE has the nucleotide sequence corresponding to NCBI Reference Sequence: NM_{—}199229.1, which is hereby incorporated by reference in its entirety. An example of a probe that hybridizes with RPE has the nucleotide sequence of SEQ ID NO: 45 as shown below:

TTTGGAGGTAATATTGGGTTGAATTCTGACTGCCCCTTTCTAGCTGGACC

EHHADH has the nucleotide sequence corresponding to NCBI Reference Sequence: NM_{—}01966.3, which is hereby incorporated by reference in its entirety. An example of a probe that hybridizes with EHHADH has the nucleotide sequence of SEQ ID NO: 46 as shown below:

CCAGCACAGGGAACTTAGGTTAGTGTGGCAAGCCTTTCCTCTTCTGGTCT

CMAH has the nucleotide sequence corresponding to NCBI Reference Sequence: NR 002174.2, which is hereby incorporated by reference in its entirety. An example of a probe that hybridizes with CMAH has the nucleotide sequence of SEQ ID NO: 47 as shown below:

CACCACCCAACTGGAAGTCATTCCTGATGTGCTGTGAGCAGAATGGGCCT

ECD has the nucleotide sequence corresponding to NCBI Reference Sequence: NM_{—}007265.2, which is hereby incorporated by reference in its entirety. An example of a probe that hybridizes with ECD has the nucleotide sequence of SEQ ID NO: 48 as shown below:

GCCCCAAATGTGGCCCTCTTGGCACAAGTCTTTGTGATCTCAGCTGTGGG

NMD3 has the nucleotide sequence corresponding to NCBI Reference Sequence: NM_{—}015938.3, which is hereby incorporated by reference in its entirety. An example of a probe that hybridizes with NMD3 has the nucleotide sequence of SEQ ID NO: 49 as shown below:

GGTAGGGAAATTAGGGTTCAGTTTATCACTGGACATTCAGGAGGCAAGTC

SLC10A7 has the nucleotide sequence corresponding to NCBI Reference Sequence: NM_{—}001029998.3, which is hereby incorporated by reference in its entirety. An example of a probe that hybridizes with SLC10A7 has the nucleotide sequence of SEQ ID NO: 50 as shown below:

CTACGAGAACACTTTTCTGTGTTTCCCCCATGCCGTCCTGTCACATCCTC

SNX4 has the nucleotide sequence corresponding to NCBI Reference Sequence: NM_{—}003794.3, which is hereby incorporated by reference in its entirety. An example of a probe that hybridizes with SNX4 has the nucleotide sequence of SEQ ID NO: 51 as shown below:

CTGCTTGGCTCTCTACACATGGCATTTCAGGGTATAAGATGTAGCATTTC

NEDD1 has the nucleotide sequence corresponding to NCBI Reference Sequence: NM_{—}152905.3, which is hereby incorporated by reference in its entirety. An example of a probe that hybridizes with NEDD1 has the nucleotide sequence of SEQ ID NO: 52 as shown below:

CTGTCACTGCTGGAGTTGCCAGTTCACTCTCAGAAAAAATAGCCGACAGC

GABPA has the nucleotide sequence corresponding to NCBI Reference Sequence: NM_{—}002040.3, which is hereby incorporated by reference in its entirety. An example of a probe that hybridizes with GABPA has the nucleotide sequence of SEQ ID NO: 53 as shown below:

GGTTTTGCACCATCCTCTTACGGCCTAGAGAGTTGACAAGTTGCTTGTAG

MAGMAS has the nucleotide sequence corresponding to NCBI Reference Sequence: NM_{—}016069.9, which is hereby incorporated by reference in its entirety. An example of a probe that hybridizes with MAGMAS has the nucleotide sequence of SEQ ID NO: 54 as shown below:

TTCTACCTGCAGTCAAAGGTGGTCCGCGCAAAGGAGCGCCTGGATGAGGA

UBE2V2 has the nucleotide sequence corresponding to NCBI Reference Sequence: NM_{—}003350.2, which is hereby incorporated by reference in its entirety. An example of a probe that hybridizes with UBE2V2 has the nucleotide sequence of SEQ ID NO: 55 as shown below:

CAAGATGAAAGCGTGTGGAGAAGTGTCAGATGGCAGTGGAAGCATGTGTG

C15ORF44 has the nucleotide sequence corresponding to NCBI Reference Sequence: NM_{—}030800.2, which is hereby incorporated by reference in its entirety. An example of a probe that hybridizes with C15ORF44 has the nucleotide sequence of SEQ ID NO: 56 as shown below:

GTGACTGTCTGGATCAAACCCAGCGGCCTGCAGACAGATGTACAGAAGAT

PCGF6 has the nucleotide sequence corresponding to NCBI Reference Sequence: NM_{—}001011663.1, which is hereby incorporated by reference in its entirety. An example of a probe that hybridizes with PCGF6 has the nucleotide sequence of SEQ ID NO: 57 as shown below:

GAGGTCTAGAAGTACCTAAACCTGCTGTTCCACAGCCAGTCCCTTCAAGC

CABIN1 has the nucleotide sequence corresponding to NCBI Reference Sequence: NM_{—}012295.3, which is hereby incorporated by reference in its entirety. An example of a probe that hybridizes with CABIN1 has the nucleotide sequence of SEQ ID NO: 58 as shown below:

TGACTTTGTAAATCTGCCCACACCCAGCTGGCCATATCCACCCCTCGACG

EIF3J has the nucleotide sequence corresponding to NCBI Reference Sequence: NM_{—}003758.2, which is hereby incorporated by reference in its entirety. An example of a probe that hybridizes with EIF3J has the nucleotide sequence of SEQ ID NO: 59 as shown below:

GCCAGGTCCTTATGTTGTCACCATAGAGCAACAAAGGTATAGGGCTGCCT

HS.561844 has the nucleotide sequence corresponding to NCBI Reference Sequence: BX101194.1, which is hereby incorporated by reference in its entirety. An example of a probe that hybridizes with HS.561844 has the nucleotide sequence of SEQ ID NO: 60 as shown below:

CAACCTTAGTTCACAGTGCAGTACCACCCCTCACCTTCCATGACTTGTCC

IMPACT has the nucleotide sequence corresponding to NCBI Reference Sequence: NM_{—}018439.2, which is hereby incorporated by reference in its entirety. An example of a probe that hybridizes with IMPACT has the nucleotide sequence of SEQ ID NO: 61 as shown below:

GGCTCGTCTTGAGCTCCTGGCCTCAATCGATCTTCCTGCCAAGGTTTTGG

ATAD2 has the nucleotide sequence corresponding to NCBI Reference Sequence: NM_{—}014109.3, which is hereby incorporated by reference in its entirety. An example of a probe that hybridizes with ATAD2 has the nucleotide sequence of SEQ ID NO: 62 as shown below:

GGCTTTGGCAATTCTTTCTCAGCCTACACCCTCACTTGTTGTGGATCATG

RGL2 has the nucleotide sequence corresponding to NCBI Reference Sequence: NM_{—}004761.3, which is hereby incorporated by reference in its entirety. An example of a probe that hybridizes with RGL2 has the nucleotide sequence of SEQ ID NO: 63 as shown below:

CAGAAATTCAGAAAGGGAGCCAGCCACCCTGGGGCAGTGAAGTGCCACTG

CASD1 has the nucleotide sequence corresponding to NCBI Reference Sequence: NM_{—}022900.4, which is hereby incorporated by reference in its entirety. An example of a probe that hybridizes with CASD1 has the nucleotide sequence of SEQ ID NO: 64 as shown below:

GCCAGTATCACATATGGCTGGCAGCGGACACAAGGGGTATCTTGGTACTG

TMEM185A has the nucleotide sequence corresponding to NCBI Reference Sequence: NM_{—}032508.2, which is hereby incorporated by reference in its entirety. An example of a probe that hybridizes with TMEM185A has the nucleotide sequence of SEQ ID NO: 65 as shown below:

ACGTGTGCGTGCCGTTTCTCCAAGCACTGCAGGTTCCACCGTGTGTCAGA

ESM1 has the nucleotide sequence corresponding to NCBI Reference Sequence: NM_{—}007036.3, which is hereby incorporated by reference in its entirety. An example of a probe that hybridizes with ESM1 has the nucleotide sequence of SEQ ID NO: 66 as shown below:

CTGCTGATGTAGTTCCCGGGTTACCTGTATCTGAAGGACGGTTCTGGGGC

ADSSL1 has the nucleotide sequence corresponding to NCBI Reference Sequence: NM_{—}152328.3, which is hereby incorporated by reference in its entirety. An example of a probe that hybridizes with ADSSL1 has the nucleotide sequence of SEQ ID NO: 67 as shown below:

AGGGCAAAGGCAAGGTGGTGGACCTGCTGGCCACGGACGCCGACATCATC

ACSL5 has the nucleotide sequence corresponding to NCBI Reference Sequence: NM_{—}203379.1, which is hereby incorporated by reference in its entirety. An example of a probe that hybridizes with ACSL5 has the nucleotide sequence of SEQ ID NO: 68 as shown below:

TTAGTAACCACAAGTTCAAGGGTCAAAGGGACCCTCTGTGCCTTCTTCTT

C1ORF124 has the nucleotide sequence corresponding to NCBI Reference Sequence: NM_{—}032018.4, which is hereby incorporated by reference in its entirety. An example of a probe that hybridizes with C1ORF124 has the nucleotide sequence of SEQ ID NO: 69 as shown below:

GCCATCCCAGGATGTGAGTGGGTCTGAAGATACATTCCCAAATAAACGAC

CYB561 has the nucleotide sequence corresponding to NCBI Reference Sequence: NM_{—}001017917.1, which is hereby incorporated by reference in its entirety. An example of a probe that hybridizes with CYB561 has the nucleotide sequence of SEQ ID NO: 70 as shown below:

GGGGCCAGTCTCCTCTAATGCTCAGATTTCCCATAGTTGGCTTTTGCTGT

MAP4K5 has the nucleotide sequence corresponding to NCBI Reference Sequence: NM_{—}006575.4, which is hereby incorporated by reference in its entirety. An example of a probe that hybridizes with MAP4K5 has the nucleotide sequence of SEQ ID NO: 71 as shown below:

GGGTTGTCGTTTTGGAAAGTAGGCCAACAGAAAATCCTACTGCACACAGC

CRIP1 has the nucleotide sequence corresponding to NCBI Reference Sequence: NM_{—}001311.4, which is hereby incorporated by reference in its entirety. An example of a probe that hybridizes with CRIP1 has the nucleotide sequence of SEQ ID NO: 72 as shown below:

GCCGAGAGCCACACTTTCAAGTAAACCAGGTGGTGGAGACCCCATCCTTG

ING1 has the nucleotide sequence corresponding to NCBI Reference Sequence: NM_{—}198219.2, which is hereby incorporated by reference in its entirety. An example of a probe that hybridizes with ING1 has the nucleotide sequence of SEQ ID NO: 73 as shown below:

GCTTGGGTACACTTCTCTTAAGTGGTCTAGTCAAGGAACCTCAAGTCATG

LILRB1 has the nucleotide sequence corresponding to NCBI Reference Sequence: XM_{—}943444.1, which is hereby incorporated by reference in its entirety. An example of a probe that hybridizes with LILRB1 has the nucleotide sequence of SEQ ID NO: 74 as shown below:

TGTCAGATGCGTCTCTGCTGACCTGAGTCTGCCCTGCACCATGGACCTGC

SPNS3 has the nucleotide sequence corresponding to NCBI Reference Sequence: NM_{—}182538.4, which is hereby incorporated by reference in its entirety. An example of a probe that hybridizes with SPNS3 has the nucleotide sequence of SEQ ID NO: 75 as shown below:

GCACATCTGCCACTTTTGAATTCCCGGCTGGAGAGCTGGCAGGACCCTGT

CDH11 has the nucleotide sequence corresponding to NCBI Reference Sequence: NM_{—}001797.2, which is hereby incorporated by reference in its entirety. An example of a probe that hybridizes with CDH11 has the nucleotide sequence of SEQ ID NO: 76 as shown below:

CGTGCCAGATATAACTGTCTTGTTTCAGTGAGAGACGCCCTATTTCTATG

LOC642403 has the nucleotide sequence corresponding to NCBI Reference Sequence: XM_{—}925924.1, which is hereby incorporated by reference in its entirety. An example of a probe that hybridizes with LOC642403 has the nucleotide sequence of SEQ ID NO: 77 as shown below:

AATGGTATGGACAGTAAGGTGCAGGCTGAGGAGGTCTCAGATGGAGATGA

CASP4 has the nucleotide sequence corresponding to NCBI Reference Sequence: NM_{—}001225.3, which is hereby incorporated by reference in its entirety. An example of a probe that hybridizes with CASP4 has the nucleotide sequence of SEQ ID NO: 78 as shown below:

ACTCCAAGGGCCAAAGCTCAAATGCCCACCATAGAACGACTGTCCATGAC

TEAD2 has the nucleotide sequence corresponding to NCBI Reference Sequence: NM_{—}003598.1, which is hereby incorporated by reference in its entirety. An example of a probe that hybridizes with TEAD2 has the nucleotide sequence of SEQ ID NO: 79 as shown below:

AGTGGGTGCTAGGGTCTTGACTTTATCTCCGCTGCACAAGCAGTGTGTTG

KHDRBS3 has the nucleotide sequence corresponding to NCBI Reference Sequence: NM_{—}006558.1, which is hereby incorporated by reference in its entirety. An example of a probe that hybridizes with KHDRBS3 has the nucleotide sequence of SEQ ID NO: 80 as shown below:

AGGCACCTTCAGCGAGGACAGCAAAGGGCGTCTACAGAGACCAGCCATAT

FHL3 has the nucleotide sequence corresponding to NCBI Reference Sequence: NM_{—}004468.4, which is hereby incorporated by reference in its entirety. An example of a probe that hybridizes with FHL3 has the nucleotide sequence of SEQ ID NO: 81 as shown below:

AGGTCTCCTATGGGTGCCTGGGAAGTCCTTGAAAGTGGACTGTTCTCAGG

EPPK1 has the nucleotide sequence corresponding to NCBI Reference Sequence: XM_{—}937579.1, which is hereby incorporated by reference in its entirety. An example of a probe that hybridizes with EPPK1 has the nucleotide sequence of SEQ ID NO: 82 as shown below:

CCAGTGTCTACTATTTAGTGCCCTGGCTCTATTTCGGTCCTCCTCCCCGG

MARCKSL1 has the nucleotide sequence corresponding to NCBI Reference Sequence: NM_{—}023009.5, which is hereby incorporated by reference in its entirety. An example of a probe that hybridizes with MARCKSL1 has the nucleotide sequence of SEQ ID NO: 83 as shown below:

CCTGAGCCAGAAGTGGGGTGCTTATACTCCCAAACCTTGAGTGTCCAGCC

FAM44B has the nucleotide sequence corresponding to NCBI Reference Sequence: NM_{—}138369.2, which is hereby incorporated by reference in its entirety. An example of a probe that hybridizes with FAM44B has the nucleotide sequence of SEQ ID NO: 84 as shown below:

TAGGCATAAGGAAACTCGTTTGCAGGCTCTCTGTCCAGGGCTGCTTCCTG

VEGFB has the nucleotide sequence corresponding to NCBI Reference Sequence: NM_{—}003377.4, which is hereby incorporated by reference in its entirety. An example of a probe that hybridizes with VEGFB has the nucleotide sequence of SEQ ID NO: 85 as shown below:

GGACAGAGTTGGAAGAGGAGACTGGGAGGCAGCAAGAGGGGTCACATACC

LYRM4 has the nucleotide sequence corresponding to NCBI Reference Sequence: NM_{—}020408.4, which is hereby incorporated by reference in its entirety. An example of a probe that hybridizes with LYRM4 has the nucleotide sequence of SEQ ID NO: 86 as shown below:

ACCGGTGCCCAAATCTGGCTGGTGGACAGAAGCACCTGGAGAGTTGGAGA

AB007962 has the nucleotide sequence corresponding to NCBI Reference Sequence: XM_{—}941243.1, which is hereby incorporated by reference in its entirety. An example of a probe that hybridizes with AB007962 has the nucleotide sequence of SEQ ID NO: 87 as shown below:

CTTATCAGAACGGAGTGGATGGTCACCTTCCTCACTTATTGCTAATCTCC

PPP2R3B has the nucleotide sequence corresponding to NCBI Reference Sequence: NM_{—}199326.1, which is hereby incorporated by reference in its entirety. An example of a probe that hybridizes with PPP2RB3 has the nucleotide sequence of SEQ ID NO: 88 as shown below:

CGTTCCTGTGAAGCTCTCCCTGACATGCATCTTCGTCTCTCCATCCTGGC

SPINK2 has the nucleotide sequence corresponding to NCBI Reference Sequence: NM_{—}021114.3, which is hereby incorporated by reference in its entirety. An example of a probe that hybridizes with SPINK2 has the nucleotide sequence of SEQ ID NO: 89 as shown below:

GCTCCTGGCAGTCACCTTCGCAGCCTCTCTGATCCCTCAATTTGGTCTGT

C9ORF123 has the nucleotide sequence corresponding to NCBI Reference Sequence: NM_{—}033428.1, which is hereby incorporated by reference in its entirety. An example of a probe that hybridizes with C9ORF123 has the nucleotide sequence of SEQ ID NO: 90 as shown below:

CGCTGAGTAGTCTGAGGAGCAAGAGGAGTTGGTCTTTCTGTCCCAGTGGC

PANK 2 has the nucleotide sequence corresponding to NCBI Reference Sequence: NM_{—}024960.4, which is hereby incorporated by reference in its entirety. An example of a probe that hybridizes with PANK2 has the nucleotide sequence of SEQ ID NO: 91 as shown below:

GCTGGCGGCCTCGACGGCAGCTGCGGAACTAGGCCGAGGGACAAAGGCTA

COG2 has the nucleotide sequence corresponding to NCBI Reference Sequence: NM_{—}007357.2, which is hereby incorporated by reference in its entirety. An example of a probe that hybridizes with COG2 has the nucleotide sequence of SEQ ID NO: 92 as shown below:

GAGAACTCCTGGGCTTTCTAAAGAGGCTGCGGGAAGCCATCCTCCACTCC

CRY2 has the nucleotide sequence corresponding to NCBI Reference Sequence: NM_{—}021117.3, which is hereby incorporated by reference in its entirety. An example of a probe that hybridizes with CRY2 has the nucleotide sequence of SEQ ID NO: 93 as shown below:

TGACCCAAGGGCCAGCATGGGGAAGAGATGGTTGCAGGCAAAATGCACTT

SESN1 has the nucleotide sequence corresponding to NCBI Reference Sequence: NM_{—}014454.2, which is hereby incorporated by reference in its entirety. An example of a probe that hybridizes with SESN1 has the nucleotide sequence of SEQ ID NO: 94 as shown below:

CCTGACACTGGAGGGCAGCTGTCTTGTGCATTACTTGTGTTCCCAGCACC

EPN2 has the nucleotide sequence corresponding to NCBI Reference Sequence: NM_{—}014964.4, which is hereby incorporated by reference in its entirety. An example of a probe that hybridizes with EPN2 has the nucleotide sequence of SEQ ID NO: 95 as shown below:

ACTGATCATGAAGCCACCGGCCACTGCCACGCATGTTGCACCTGTGCCAT

IL23A has the nucleotide sequence corresponding to NCBI Reference Sequence: X00437.1, which is hereby incorporated by reference in its entirety. An example of a probe that hybridizes with IL23A has the nucleotide sequence of SEQ ID NO: 96 as shown below:

GGAGCTTCTAACCCGTCATGGTTCAATACACATTCTTCTTTTGCCAGCGC

BE439556 has the nucleotide sequence corresponding to NCBI Reference Sequence: BE439556.1, which is hereby incorporated by reference in its entirety. An example of a probe that hybridizes with BE439556 has the nucleotide sequence of SEQ ID NO: 97 as shown below:

CCCTCTCCCTGGGGCATGTGCAGAGTTTCAACTTCTTGTTGGCTGCTCTC

DB050967 has the nucleotide sequence corresponding to NCBI Reference Sequence: DA760637.1, which is hereby incorporated by reference in its entirety. An example of a probe that hybridizes with DB050967 has the nucleotide sequence of SEQ ID NO: 98 as shown below:

CAGGACACTCTCCAAACTCTCCCTCCAGTCTAAGATGATGCCAGCTCCAG

TMEM203 has the nucleotide sequence corresponding to NCBI Reference Sequence: NM_{—}053045.1, which is hereby incorporated by reference in its entirety. An example of a probe that hybridizes with TMEM203 has the nucleotide sequence of SEQ ID NO: 99 as shown below:

GAACTCTGCTGTCCAGGCACTGCTTGGCTTACTATCCCAGCAAAGACTGC

RCBTB2 has the nucleotide sequence corresponding to NCBI Reference Sequence: NM_{—}001268.2, which is hereby incorporated by reference in its entirety. An example of a probe that hybridizes with RCBTB2 has the nucleotide sequence of SEQ ID NO: 100 as shown below:

TCACATAAGGTCCTTTGCTTTTCTTTGTGTTAAGAGGGACTTGCCTCTGT

ZNF627 has the nucleotide sequence corresponding to NCBI Reference Sequence: NM_{—}145295.2, which is hereby incorporated by reference in its entirety. An example of a probe that hybridizes with ZNF627 has the nucleotide sequence of SEQ ID NO: 101 as shown below:

GTGCTCTCTTGCTAAAGACCGCAGAGACCATACCTGTTGTCAAAGAGGGT

CMTM1 has the nucleotide sequence corresponding to NCBI Reference Sequence: NM_{—}181289.2, which is hereby incorporated by reference in its entirety. An example of a probe that hybridizes with CMTM1 has the nucleotide sequence of SEQ ID NO: 102 as shown below:

TGTACCCAAGGCACAGCGCAACATCTCAGCGAAGACCGCACCCCGGAAGC

HSD11B1L has the nucleotide sequence corresponding to NCBI Reference Sequence: NM_{—}198707.2, which is hereby incorporated by reference in its entirety. An example of a probe that hybridizes with HSD11B1L has the nucleotide sequence of SEQ ID NO: 103 as shown below:

GGGGACTTGCAAGGCCTCACCTGTTTGGCCATGATTGATGACGTGACTGC

MAL has the nucleotide sequence corresponding to NCBI Reference Sequence: NM_{—}002371.3, which is hereby incorporated by reference in its entirety. An example of a probe that hybridizes with MAL has the nucleotide sequence of SEQ ID NO: 104 as shown below:

ACTTGAGCTGAAAACCCAGATGGTGTTAACTGGCCGCCCCACTTTCCGGC

TOP1MT has the nucleotide sequence corresponding to NCBI Reference Sequence: NM_{—}052963.2, which is hereby incorporated by reference in its entirety. An example of a probe that hybridizes with TOP1MT has the nucleotide sequence of SEQ ID NO: 105 as shown below:

CAGGATCAGCATTGCCTGGTGCAAGCGGTTCAGGGTGCCAGTGGAGAAGA

NSUN5 has the nucleotide sequence corresponding to NCBI Reference Sequence: NM_{—}018044.3, which is hereby incorporated by reference in its entirety. An example of a probe that hybridizes with NSUN5 has the nucleotide sequence of SEQ ID NO: 106 as shown below:

ACGTGCTCCCTCTGCCAGGAGGAGAATGAAGACGTGGTGCGAGATGCGCT

GRB10 has the nucleotide sequence corresponding to NCBI Reference Sequence: NM_{—}001001555.2, which is hereby incorporated by reference in its entirety. An example of a probe that hybridizes with GRB10 has the nucleotide sequence of SEQ ID NO: 107 as shown below:

GCGCTCGGAGACCCGGTGGAGCCCAAAGTTTCCGCGCAGCCCCTGGGTGG

ANXA8L1 has the nucleotide sequence corresponding to NCBI Reference Sequence: XM_{—}931775.1, which is hereby incorporated by reference in its entirety. An example of a probe that hybridizes with ANXA8L1 has the nucleotide sequence of SEQ ID NO: 108 as shown below:

GGCTGTGGCCAACACTACGTGAGCCCCTCCCTTCCCAGTGGGCACCATGT

ERI2 has the nucleotide sequence corresponding to NCBI Reference Sequence: XM_{—}001132328.1, which is hereby incorporated by reference in its entirety. An example of a probe that hybridizes with ERI2 has the nucleotide sequence of SEQ ID NO: 109 as shown below:

TTACTGCCAGCATCTCAGCCTGAGGAAAACGTAGACTGTACAGTTCCCAT

AK098672 has the nucleotide sequence corresponding to NCBI Reference Sequence: XM_{—}933327.1, which is hereby incorporated by reference in its entirety. An example of a probe that hybridizes with AK098672 has the nucleotide sequence of SEQ ID NO: 110 as shown below:

GCAGAAAGTTGAGGCTTAGAAGGTGCACGGCCCTACCTCTTCCGAGGTGC

CRCP has the nucleotide sequence corresponding to NCBI Reference Sequence: NM_{—}014478.4, which is hereby incorporated by reference in its entirety. An example of a probe that hybridizes with CRCP has the nucleotide sequence of SEQ ID NO: 111 as shown below:

GAAAGATTTCCTCCACGGCCTTTGCCCCAGTTGTGGGGAGGTCTCTGTGC

TWIST2 has the nucleotide sequence corresponding to NCBI Reference Sequence: NM_{—}057179.2, which is hereby incorporated by reference in its entirety. An example of a probe that hybridizes with TWIST2 has the nucleotide sequence of SEQ ID NO: 112 as shown below:

GCAGAGCGACGAGATGGACAATAAGATGACCAGCTGCAGCTACGTGGCCC

RIMKLB has the nucleotide sequence corresponding to NCBI Reference Sequence: NM_{—}020734.2, which is hereby incorporated by reference in its entirety. An example of a probe that hybridizes with RIMKLB has the nucleotide sequence of SEQ ID NO: 113 as shown below:

GTAAATGCACCGGTTTGGATTCAGGCACAGCCCCAGTCTGCCTACAGCAG

AM393854 has the nucleotide sequence corresponding to NCBI Reference Sequence: NM_{—}133477.2, which is hereby incorporated by reference in its entirety. An example of a probe that hybridizes with AM393854 has the nucleotide sequence of SEQ ID NO: 114 as shown below:

TCCAGTAGCTGATTACAACTACAACCCACACCCAAGGGGATGGAGACGCC

PAQR6 has the nucleotide sequence corresponding to NCBI Reference Sequence: NM_{—}024897.3, which is hereby incorporated by reference in its entirety. An example of a probe that hybridizes with PAQR6 has the nucleotide sequence of SEQ ID NO: 115 as shown below:

GCCTTGAGCTCAGAGGGGGTACCCAGGCGGGCAGGGACCGTCCAGGCCCA

GTF3C6 has the nucleotide sequence corresponding to NCBI Reference Sequence: NM_{—}138408.3, which is hereby incorporated by reference in its entirety. An example of a probe that hybridizes with GTF3C6 has the nucleotide sequence of SEQ ID NO: 116 as shown below:

CGGAGAAGACGAGGAAGAGGAGGAGCAGTTGGTTCTGGTGGAATTATCAG

GRASP has the nucleotide sequence corresponding to NCBI Reference Sequence: NM_{—}181711.3, which is hereby incorporated by reference in its entirety. An example of a probe that hybridizes with GRASP has the nucleotide sequence of SEQ ID NO: 117 as shown below:

TCCCATGAAGCCCTCTCCTCAGCTTTACTTGCTCCCCCGCCCTTAGCCTT

CENPE has the nucleotide sequence corresponding to NCBI Reference Sequence: NM_{—}001813.2, which is hereby incorporated by reference in its entirety. An example of a probe that hybridizes with CENPE has the nucleotide sequence of SEQ ID NO: 118 as shown below:

CCAGAGGTGCAAAATGCAGGAGCAGAGAGTGTGGATTCTCAGCCAGGTCC

P2RY4 has the nucleotide sequence corresponding to NCBI Reference Sequence: NM_{—}002565.4, which is hereby incorporated by reference in its entirety. An example of a probe that hybridizes with P2RY4 has the nucleotide sequence of SEQ ID NO: 119 as shown below:

CTTGCTCACTGGGGACAAATATCGACGTCAGCTCCGTCAGCTCTGTGGTG

BC038536 has the nucleotide sequence corresponding to NCBI Reference Sequence: XM_{—}931524.1, which is hereby incorporated by reference in its entirety. An example of a probe that hybridizes with BC038536 has the nucleotide sequence of SEQ ID NO: 120 as shown below:

GGGCTAACACATTAATGAGATTTCCTGAGACTGCCCTGTAGAGATGCTTG

ZNF268 has the nucleotide sequence corresponding to NCBI Reference Sequence: NM_{—}152943.2, which is hereby incorporated by reference in its entirety. An example of a probe that hybridizes with ZNF268 has the nucleotide sequence of SEQ ID NO: 121 as shown below:

GTTTGGTGTCTGGTGAGGGCCTGCTCTGTGCTTCCAAGATGACGCCTTGT

SMPD1 has the nucleotide sequence corresponding to NCBI Reference Sequence: NM_{—}001007593.2, which is hereby incorporated by reference in its entirety. An example of a probe that hybridizes with SMPD1 has the nucleotide sequence of SEQ ID NO: 122 as shown below:

CTCCTTTCCTGGAGCTGGTTTAGCTGGATATGGGAGGGGGTTTGGCTGCC

MRP63 has the nucleotide sequence corresponding to NCBI Reference Sequence: NM_{—}024026.4, which is hereby incorporated by reference in its entirety. An example of a probe that hybridizes with MRP63 has the nucleotide sequence of SEQ ID NO. 123 as shown below.

GTCAAAGTTGTATGGCTGAGGCCCACACGGTGGCTCACTTCTGTAATCCC

CSTF3 has the nucleotide sequence corresponding to NCBI Reference Sequence: NM_{—}001326.2, which is hereby incorporated by reference in its entirety. An example of a probe that hybridizes with CSTF3 has the nucleotide sequence of SEQ ID NO: 124 as shown below:

GTGGCAGAACCACATTTTGTTCCCTCTTCAAGGGTGTCTTGTATGTGCCG

TTF2 has the nucleotide sequence corresponding to NCBI Reference Sequence: NM_{—}003594.3, which is hereby incorporated by reference in its entirety. An example of a probe that hybridizes with TTF2 has the nucleotide sequence of SEQ ID NO: 125 as shown below:

CAGCCATCTCTGCAGTTCTCTCAGTGCAGGCAGTTCTTCCTCTCAGGCTG

AW004814 has the nucleotide sequence corresponding to NCBI Reference Sequence: AW004814.1, which is hereby incorporated by reference in its entirety. An example of a probe that hybridizes with AW004814 has the nucleotide sequence of SEQ ID NO: 126 as shown below:

AAAGAATGACCCCTGTTGATGAGAGGTGTTACATGTGGCCATGGGACAGG

AW119108 has the nucleotide sequence corresponding to NCBI Reference Sequence: AW119108.1, which is hereby incorporated by reference in its entirety. An example of a probe that hybridizes with AW119108 has the nucleotide sequence of SEQ ID NO: 127 as shown below:

CACGGTCTGGAAGGTGGAAAGTCCAAGATCATGGCTCTGGCAGGTCTCTG

AW182429 has the nucleotide sequence corresponding to NCBI Reference Sequence: AW182429.1, which is hereby incorporated by reference in its entirety. An example of a probe that hybridizes with AW182429 has the nucleotide sequence of SEQ ID NO: 128 as shown below:

GGCTTCAGAGCTGTATTTCGGCCAACCCAGTCTTTGTTCAGTTTCTAGCG

HS.566857 has the nucleotide sequence corresponding to NCBI Reference Sequence: AI83821.1, which is hereby incorporated by reference in its entirety. An example of a probe that hybridizes with HS.566857 has the nucleotide sequence of SEQ ID NO: 129 as shown below:

TTGCAGGGGGCAGACAGATTTATTATGAGGAATTGACTCCTGTGATTATG

BX109554 has the nucleotide sequence corresponding to NCBI Reference Sequence: BX109554.1, which is hereby incorporated by reference in its entirety. An example of a probe that hybridizes with BX109554 has the nucleotide sequence of SEQ ID NO: 130 as shown below:

GCCTGTGAGATGGGTTGCAGAATTGACCTGAGCAGACCAGGCTTCCGAGG

APOBEC3B has the nucleotide sequence corresponding to NCBI Reference Sequence: NM_{—}004900.4, which is hereby incorporated by reference in its entirety. An example of a probe that hybridizes with APOBEC3B has the nucleotide sequence of SEQ ID NO: 131 as shown below:

ACCCTTTGGTCCTTCGACGGCGCCAGACCTACTTGTGCTATGAGGTGGAG

EZH2 has the nucleotide sequence corresponding to NCBI Reference Sequence: NM_{—}152998.2, which is hereby incorporated by reference in its entirety. An example of a probe that hybridizes with EZH2 has the nucleotide sequence of SEQ ID NO: 132 as shown below:

AGTGTGACCCTGACCTCTGTCTTACTTGTGGAGCCGCTGACCATTGGGAC

GCOM1 has the nucleotide sequence corresponding to NCBI Reference Sequence: NM_{—}015532.3, which is hereby incorporated by reference in its entirety. An example of a probe that hybridizes with GCOM1 has the nucleotide sequence of SEQ ID NO: 133 as shown below:

ATTGCAGAATGTGAAGAAGTTAGAAGAAAAAGTGAACTGTTTAACCCTGT

ZBTB20 has the nucleotide sequence corresponding to NCBI Reference Sequence: XM_{—}932718.1, which is hereby incorporated by reference in its entirety. An example of a probe that hybridizes with ZBTB20 has the nucleotide sequence of SEQ ID NO: 134 as shown below:

TGTTTTCTTAACTTCCTAACCTATGGTTAATTATTCTGACCAGTGGAAAG

LOC729973 has the nucleotide sequence corresponding to NCBI Reference Sequence: XM_{—}001131929.1, which is hereby incorporated by reference in its entirety. An example of a probe that hybridizes with LOC729973 has the nucleotide sequence of SEQ ID NO: 135 as shown below:

GAGGAGCCATAGCAGCAAGGAGTACCTGGAGCTGCACAGGGAGAACTTCC

MAP2K3 has the nucleotide sequence corresponding to NCBI Reference Sequence: NM_{—}002756.4, which is hereby incorporated by reference in its entirety. An example of a probe that hybridizes with MAP2K3 has the nucleotide sequence of SEQ ID NO: 136 as shown below:

GGGACCTTTGGAGCACAGCCTACGATCCTGGTGCAAGGCCGGTGGATGCA

BF701780 has the nucleotide sequence corresponding to NCBI Reference Sequence: XM_{—}932199.1, which is hereby incorporated by reference in its entirety. An example of a probe that hybridizes with BF701780 has the nucleotide sequence of SEQ ID NO: 137 as shown below:

AGCACTGTCGAGGTCACAGACTGCAGACCTCCTTGTATCTTCAAATGGAG

KCNA6 has the nucleotide sequence corresponding to NCBI Reference Sequence: NM_{—}002235.3, which is hereby incorporated by reference in its entirety. An example of a probe that hybridizes with KCNA6 has the nucleotide sequence of SEQ ID NO: 138 as shown below:

CCCCTCCCTACCTCATGGGGAATGTCTGGGAAGCTGGGGACATTGCTATG

OR1J4 has the nucleotide sequence corresponding to NCBI Reference Sequence: NM_{—}001004452.1, which is hereby incorporated by reference in its entirety. An example of a probe that hybridizes with OR1J4 has the nucleotide sequence of SEQ ID NO: 139 as shown below:

GGCCACATTGGGGTCACCATCCTCAAGGCTCCATCTACTAAGGGCATCTT

SKP1 has the nucleotide sequence corresponding to NCBI Reference Sequence: XM_{—}940504.1, which is hereby incorporated by reference in its entirety. An example of a probe that hybridizes with SKP1 has the nucleotide sequence of SEQ ID NO: 140 as shown below:

TTGAAGTTGATGTGGAAATTGCCAAACAATCTGTGACTCTCAAAATCATG

STAT1 has the nucleotide sequence corresponding to NCBI Reference Sequence: NM_{—}007315.2, which is hereby incorporated by reference in its entirety. An example of a probe that hybridizes with STAT1 has the nucleotide sequence of SEQ ID NO: 141 as shown below:

TACTCCAGGCCAAAGGAAGCACCAGAGCCAATGGAACTTGATGGCCCTAA

STAT1 also has the nucleotide sequence corresponding to NCBI Reference Sequence: NM_{—}139266.2, which is hereby incorporated by reference in its entirety. An example of a probe that hybridizes with STAT1 has the nucleotide sequence of SEQ ID NO: 142 as shown below:

CGCCATCACAGCTGAACTTGTTGAGATCCCCGTGTTACTGCCTATCAGCA

C1 ORF26 has the nucleotide sequence corresponding to NCBI Reference Sequence: NM_{—}017673.6, which is hereby incorporated by reference in its entirety. An example of a probe that hybridizes with C1ORF26 has the nucleotide sequence of SEQ ID NO: 143 as shown below:

GGGCTTGTCACTTTTCAGTACATTTCTAGTGGGGAAGAGCAGGGCGACCC

VAT1 has the nucleotide sequence corresponding to NCBI Reference Sequence: NM_{—}006373.3, which is hereby incorporated by reference in its entirety. An example of a probe that hybridizes with VAT1 has the nucleotide sequence of SEQ ID NO: 144 as shown below:

AGGACCTGGGCCATTGCAACCAAAATGGGGACTTCCTGGGTAGGGAGGTC

LOC390427 has the nucleotide sequence corresponding to NCBI Reference Sequence: XM_{—}939524.1, which is hereby incorporated by reference in its entirety. An example of a probe that hybridizes with LOC390427 has the nucleotide sequence of SEQ ID NO: 145 as shown below:

CCACGTTTCTGTGACTTGAGTGCCCTGGGAGTTTTACGCTGCCTCGTCTC

THSD1 has the nucleotide sequence corresponding to NCBI Reference Sequence: NM_{—}018676.3, which is hereby incorporated by reference in its entirety. An example of a probe that hybridizes with THSD1 has the nucleotide sequence of SEQ ID NO: 146 as shown below:

GAGCATTTCCAAGAGGCAAGTGGAACCCGTGGTCCATTAAACCCTCTCCC

C70RF49 has the nucleotide sequence corresponding to NCBI Reference Sequence: NM_{—}024033.2, which is hereby incorporated by reference in its entirety. An example of a probe that hybridizes with C70RF49 has the nucleotide sequence of SEQ ID NO: 147 as shown below:

GCAAGAGGGACAGGAGCCCAGAAGAGACACTGAGGACAAGAGATCACACC

SSX5 has the nucleotide sequence corresponding to NCBI Reference Sequence: XM_{—}941508.1, which is hereby incorporated by reference in its entirety. An example of a probe that hybridizes with SSX5 has the nucleotide sequence of SEQ ID NO: 148 as shown below:

CTCTGCCTTCCCAACAAGGGTCTGTACATCTTTCAGGGACAGACTGCTCC

TMPRSS11B has the nucleotide sequence corresponding to NCBI Reference Sequence: NM_{—}182502.3, which is hereby incorporated by reference in its entirety. An example of a probe that hybridizes with TMPRSS11B has the nucleotide sequence of SEQ ID NO: 149 as shown below:

GAGGTCATCAAGGGCAGAGAACTGTGTGGCATCTCCTTATAGTAAAAGTG

DIP2B has the nucleotide sequence corresponding to NCBI Reference Sequence: NM_{—}173602.2, which is hereby incorporated by reference in its entirety. An example of a probe that hybridizes with DIP2B has the nucleotide sequence of SEQ ID NO: 150 as shown below:

CAGGCTGTTTAGGGACCATTGCCTGTCTTGGTCACATGAGTCTGTCTCCT

RFX3 has the nucleotide sequence corresponding to NCBI Reference Sequence: NM_{—}134428.2, which is hereby incorporated by reference in its entirety. An example of a probe that hybridizes with RFX3 has the nucleotide sequence of SEQ ID NO: 151 as shown below:

GGCGTGCAACCAAGCCTCCTGAATCCAATTCACAGCGAGCACATTGTCAC

ZNF774 has the nucleotide sequence corresponding to NCBI Reference Sequence: NM_{—}001004309.2, which is hereby incorporated by reference in its entirety. An example of a probe that hybridizes with ZNF774 has the nucleotide sequence of SEQ ID NO: 152 as shown below:

TGCTACTGTCTTCAAGCACCCCAAATAGAGAAAACCTGGGCGTCAGTGGC

GPAH2 has the nucleotide sequence corresponding to NCBI Reference Sequence: NM_{—}130769.3, which is hereby incorporated by reference in its entirety. An example of a probe that hybridizes with GPAH2 has the nucleotide sequence of SEQ ID NO: 153 as shown below:

AAAGTACAGCTGCAGTGTGTGGGGAGCCGGAGGGAGGAGCTCGAGATCTT

RDH11 has the nucleotide sequence corresponding to NCBI Reference Sequence: NM_{—}016026.3, which is hereby incorporated by reference in its entirety. An example of a probe that hybridizes with RDH11 has the nucleotide sequence of SEQ ID NO: 154 as shown below:

GGAGGAATTGAGGGCAAGCACCCAGGACTGATGAGGTCTTAACAAAAACC

BC050625 has the nucleotide sequence corresponding to NCBI Reference Sequence: NM_{—}052941.4, which is hereby incorporated by reference in its entirety. An example of a probe that hybridizes with BC050625 has the nucleotide sequence of SEQ ID NO: 155 as shown below:

CCATGGGCCTTTTCACAGGGGACACAGGCTTCTTAAAACAACCCGGCTTC

DBF4 has the nucleotide sequence corresponding to NCBI Reference Sequence: NM_{—}006716.4, which is hereby incorporated by reference in its entirety. An example of a probe that hybridizes with DBF4 has the nucleotide sequence of SEQ ID NO: 156 as shown below:

GACTCTACCCCCTAATTTGGTAGGAGATGAAGGAGAAAAGGATGGCATTG

BX248296 has the nucleotide sequence corresponding to NCBI Reference Sequence: XM_{—}375081.3, which is hereby incorporated by reference in its entirety. An example of a probe that hybridizes with BX248296 has the nucleotide sequence of SEQ ID NO: 157 as shown below:

CTGCTACTTGACCCTAGAGCTCTTTGACTCCGATTCCCATGTACGTGCCC

CD364714 has the nucleotide sequence corresponding to NCBI Reference Sequence: CD364714, which is hereby incorporated by reference in its entirety. An example of a probe that hybridizes with CD364714 has the nucleotide sequence of SEQ ID NO: 158 as shown below:

GCAAGGCTGGGGTGCTTTGAGATTGTCCATGTCTGATCTACGCACCACGC

DA196703 has the nucleotide sequence corresponding to NCBI Reference Sequence: DA196703, which is hereby incorporated by reference in its entirety. An example of a probe that hybridizes with DA196703 has the nucleotide sequence of SEQ ID NO: 159 as shown below:

GGGGGAGGGAGCGTGAAGAATTGTTTAACATTTGGCCATCGTTTGGCTGC

AA884785 has the nucleotide sequence corresponding to NCBI Reference Sequence: AA884785, which is hereby incorporated by reference in its entirety. An example of a probe that hybridizes with AA884785 has the nucleotide sequence of SEQ ID NO: 160 as shown below:

CCCTCAGAGTCAGTGATCCAAGAGTGTAAGCTACCAGTGCTTCCTGGGAC

ZNF33B has the nucleotide sequence corresponding to NCBI Reference Sequence: BI258188.1, which is hereby incorporated by reference in its entirety. An example of a probe that hybridizes with ZNF33B has the nucleotide sequence of SEQ ID NO: 161 as shown below:

GCTGGAAATGTGGGGGCAACAAATACATTAGTGAACACCCTGGCGGATCC

AK125234 has the nucleotide sequence corresponding to NCBI Reference Sequence: AK125234, which is hereby incorporated by reference in its entirety. An example of a probe that hybridizes with AK125234 has the nucleotide sequence of SEQ ID NO: 162 as shown below:

GAGGCCCTGGAGGATGGCAGAGGGAACAGGAGGAAAACAAAGTCACATGC

AA961268 has the nucleotide sequence corresponding to NCBI Reference Sequence: AA961268, which is hereby incorporated by reference in its entirety. An example of a probe that hybridizes with AA961268 has the nucleotide sequence of SEQ ID NO: 163 as shown below:

ACCACCAGCTTTCCTGGTTCTCCACCTTGCAGAAAGAAGATCCTGGGACT

LGSN has the nucleotide sequence corresponding to NCBI Reference Sequence: NM_{—}016571.2, which is hereby incorporated by reference in its entirety. An example of a probe that hybridizes with LGSN has the nucleotide sequence of SEQ ID NO: 164 as shown below:

GCAAACTTACCAGATCTTGTCAGTCATTTCCTATGTGTATGTTGACCTGG

STAG3L1 has the nucleotide sequence corresponding to NCBI Reference Sequence: NM_{—}001002840.1, which is hereby incorporated by reference in its entirety. An example of a probe that hybridizes with STAG3L1 has the nucleotide sequence of SEQ ID NO: 165 as shown below:

TGGCCAGGATGGTCTTGATCTCTTGACCTTGTGATCCACCTGCCTCATCA

One of skill in the art will appreciate that probe sequences, other than those disclosed above, that hybridize to the target sequence or isoforms of the target sequence are encompassed by the present invention.

As used herein, the term “biomarker” means an entire gene, or a portion thereof, such as an EST derived from that gene, the expression or level of which changes between certain conditions. When the expression of the gene correlates with a certain condition, for example a drug treatment or a disease state (i.e. an ASD), the gene is a marker for that condition.

As used herein, the term “probe” refers to a nucleic acid molecule, or oligonucleotide, corresponding to DNA or RNA, with a variable length that is complementary to a portion of the biomarkers of the present invention.

Probes of the present invention can be naturally occurring or synthetic, but are typically prepared by synthetic means. Synthesis of probes is well known to those skilled in the art (Oligonucleotide Synthesis: Methods and Applications, Piet Herdewijn (ed) Humana Press (2004), which is hereby incorporated by reference in its entirety.) For the purpose of the present invention, a probe comprises an oligonucleotide capable of hybridizing specifically to a nucleic acid target molecule of a complementary sequence through one or more types of chemical bond. Such binding may usually occur through complementary base pairing, and usually through hydrogen bond formation. Suitable probes may include natural (i.e. A, G, C, or T) or modified bases (7-deazaguanosine, inosine, etc.). In addition, a linkage other than a phosphodiester bond may be used to join the bases in the probe, so long as this variation does not interfere with hybridization of the probe to its target. Thus, probes of the present invention may be peptide nucleic acids in which the constituent bases are joined by peptide bonds rather than phosphodiester linkages. The minimum size of the probes of the present invention is the size required for formation of a stable hybrid between the probe and a complementary sequence on a nucleic acid molecule.

The phrase “hybridizing specifically to” as used herein refers to the binding, duplexing, or hybridizing of an oligonucleotides probe preferentially to a particular target nucleotide sequence under stringent conditions when that sequence is present in a sample. Stringent conditions are sequence-dependent and will be different in different circumstances. For example, longer sequences hybridize specifically at higher temperatures.

The probes or targets of the present invention may also be labeled in order that they may be easily detected. Examples of detectable moieties that may be used in the labeling of probes or targets include various enzymes, prosthetic groups, fluorescent materials, luminescent materials, bioluminescent materials, radioactive materials and colorimetric materials.

The present invention is also directed to a method of diagnosing whether a subject has an ASD that involves obtaining a biological sample from a subject potentially having an ASD and providing a collection of probes recognizing biomarkers comprising at least 50% of the biomarkers from one of the following biomarker sets: (1) ZNF329, LOC641518, TAP1, GBP2, RAB3IP, and MYOF; (2) MRPS10, ARF3, CLORF85, KCNE1L, BIN2, CACHD1, CYB5R3, FKBP12-EXI, CHM, DUS4L, STX5, AK3, BU580973, TCRA, CR608770, and SPI1; (3) SC65, FUNDC2, NDRG2, RPL28, SRP54, LOC643466, ZDHHC11B, NSUN5B, NDRG3, DHRS3, CPEB2, RAB3IP, PPID, FOXP1, and EFNA1; or (4) C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, and MAP4K5. The biological sample is then contacted with said collection of probes under conditions effective to permit hybridization of said probes to complementary nucleic acid molecules, if present, in the sample. This method further involves detecting any hybridization as a result of said contacting, and identifying whether or not the subject has an ASD based on said detecting.

The method described above will identify whether or not a child who is already displaying developmental concerns actually has an ASD or will go on to develop normally. Also, as shown in the Examples, this method can also distinguish the diagnosis of ASD from other developmental disorders, specifically developmental delay (DD) and language delay (LD).

As described herein, a collection of probes recognizing at least 50% of the following biomarkers: ZNF329, LOC641518, TAP1, GBP2, RAB3IP, and MYOF is informative of a diagnosis of an ASD when there is an increase in expression levels of ZNF329, LOC641518, and RAB31P, along with a corresponding decrease in TAP1, GBP2, and MYOF, as compared to a normally developing subject.

This collection of probes is most effective for diagnosing an ASD when used with subjects between 12 and 24 months of age that are also matched on gender and age within 1 month.

The collection of probes recognizing at least 50% of the following biomarkers: MRPS10, ARF3, CLORF85, KCNE1L, BIN2, CACHD1, CYB5R3, FKBP12-EXI, CHM, DUS4L, STX5, AK3, BU580973, TCRA, CR608770, and SPI1 is informative of a diagnosis of an ASD when there is an increase in the expression levels of MRPS10, CACHD1, CHM, DUS4L, AK3, BU580973, TCRA, and CR608770, along with a corresponding decrease in ARF3, C1ORF85, KCNE1L, BIN2, CYB5R3, FKBP12-EXI, STX5, and SPI1, as compared to a subject with a DD or an LD.

This collection of probes is most effective for diagnosing an ASD when used with subjects between 12 and 24 months of age that are also matched on gender and age within 1 month.

The collection of probes recognizing at least 50% of the following biomarkers: SC65, FUNDC2, NDRG2, RPL28, SRP54, LOC643466, ZDHHC11B, NSUN5B, NDRG3, DHRS3, CPEB2, RAB3IP, PPID, FOXP1, and EFNA1 is informative of a diagnosis of an ASD, DD, or LD when there is an increase in the expression levels of SC65, FUNDC2, NDRG2, RPL28, LOC643466, NSUN5B, NDRG3, DHRS3, CPEB2, FOXP1, EFNA1 and RAB3IP, along with a corresponding decrease in SRP54, ZDHHC11B, and PPID, as compared to a normally developing subject.

This collection of probes is most effective for diagnosing an ASD when used with subjects between 12 and 24 months of age that are also matched on gender and age within 1 month.

The collection of probes recognizing at least 50% of the following biomarkers: C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, and MAP4K5 is informative of a diagnosis of an ASD when there is an increase in the expression levels of CR617556, MAGMAS, CABIN1, HS.561844, RGL2, and TMEM185A along with a corresponding decrease in C5ORF44, ARHGAP25, CTDSPL2, CKAP2, MAZ, BET1, SRP54, RPE, EHHADH, CMAH, ECD, NMD3, SLC10A7, SNX4, NEDD1, GABPA, UBE2V2, GBP2, C15ORF44, PCGF6, EIF3J, IMPACT, ATAD2, ESM1, ADSSL1, ACSL5, C1ORF124, CYB561, and MAP4K5, as compared to a normally developing subject.

This collection is also informative of a diagnosis of ASD as compared to a subject with DD or LD when there is an increase in expression levels of C5ORF44, CTDSPL2, CKAP2, BET1, SRP54, CR617556, RPE, EHHADH, CMAH, ECD, NMD3, SLC10A7, SNX4, NEDD1, GABPA, UBE2V2, C15ORF44, PCGF6, EIF3J, HS.561844, IMPACT, ATAD2, CASD1, ESM1, ADSSL1, ACSL5, C1ORF124, CYB561, MAP4K5, and a corresponding decrease in ARHGAP25, MAZ, MAGMAS, CABIN1, RGL2, TMEM185A.

This collection of probes is most effective for diagnosing an ASD when used with subjects between 12 and 24 months of age that are also matched on gender and age within 1 month.

Another aspect of the present invention is directed to a method of determining whether a subject has a predisposition for developing an ASD. This method involves obtaining a biological sample from a subject at risk of potentially having a predisposition for developing an ASD and providing a collection of probes recognizing biomarkers comprising at least 50% of the biomarkers from one of the following biomarker sets: (1) CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, and NSUN5; (2) GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, and EFNA1; or (3) GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1. The biological sample from the subject is then contacted with said collection of probes under conditions effective to permit hybridization of said probes to complementary nucleic acid molecules, if present, in the sample. This method further involves detecting any hybridization as a result of said contacting, and identifying whether or not the subject has a predisposition for developing an ASD based on said detecting.

As described herein, the collection of probes recognizing at least 50% of the following biomarkers: CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, and NSUN5 is informative for a subject having a predisposition for developing an ASD when there is a increase in expression levels of CRIP1, ING1, SPNS3, CDH11, LOC642403, TEAD2, KHDRBS3, FHL3, LOC641518, EPPK1, MARCKSL1, FAM44B, VEGFB, LYRM4, PPP2R3B, SPINK2, C9ORF123, COG2, CRY2, SESN1, EPN2, IL23A, BE439556, DB050967, TMEM203, ZNF627, HSD11B1L, MAL, TOP1MT, and NSUN5 along with a corresponding decrease in LILRB1, CASP4, AB007962, PANK2, RCBTB2, and CMTM1, as compared to a normally developing subject.

This collection of probes is most effective for determining that a subject has a predisposition for developing an ASD when used with subjects between 12 and 24 months of age that are also matched on gender and age within 1 month.

The collection of probes recognizing at least 50% of the following biomarkers: GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, and EFNA1 is informative for a subject having a predisposition for developing an ASD, DD, or LD when there is a increase in expression levels of GRB10, ANXA8L1, AK098672, RIMKLB, PAQR6, GRASP, SMPD1, AW004814, AW119108, HS.566857, BX109554, and EFNA along with a corresponding decrease in ERI2, CRCP, TWIST2, AM393854, GTF3C6, CENPE, P2RY4, BC038536, ZNF268, PANK2, MRP63, CSTF3, TTF2, AW182429, ATAD2, ESM1, APOBEC3B, EZH2, ACSL5, as compared to a normally developing subject.

This collection of probes is most effective for determining that a subject has a predisposition for developing an ASD, DD, or LD when used with subjects between 12 and 24 months of age that are also matched on gender and age within 1 month.

The collection of probes recognizing at least 50% of the following biomarkers: GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1 is informative for a subject having a predisposition for developing an ASD when there is a increase in expression levels of ZBTB20, BF701780, RIMKLB, OR1J4, SKP1, VAT1, LOC390427, C7ORF49, SSX5, TMPRSS11B, ZNF774, RDH11, DBF4, BX248296, RAB3IP, CD364714, DA196703, AA884785, ZNF33B, and AK125234 along with a corresponding decrease in LOC729973, MAP2K3, KCNA6, STAT1, C1ORF26, THSD1, DIP2B, RFX3, GPHA2, BC050625, AA961268, LGSN, and STAG3L1, as compared to a normally developing subject.

This collection is also informative of a diagnosis of ASD as compared to a subject with DD or LD when there is an increase in expression levels of ZBTB20, BF701780, RIMKLB, KCNA6, OR1J4, SKP1, C1ORF26, VAT1, LOC390427, C7ORF49, TMPRSS11B, ZNF774, RDH11, DBF4, BX248296, RAB3IP, CD364714, DA196703, AA884785, ZNF33B, and AK125234 along with a corresponding decrease in GCOM1, LOC729973, MAP2K3, STAT1, THSD1, DIP2B, RFX3, GPHA2, BC050625, AA961268, LGSN, and STAG3L1 and no change in SSX5.

This collection of probes is most effective for determining that a subject has a predisposition for developing an ASD when used with subjects between 12 and 24 months of age that are also matched on gender and age within 1 month.

The present invention is also directed to a method of diagnosing whether a subject has an autism spectrum disorder involving obtaining a biological sample from a subject potentially having an autism spectrum disorder, providing one or more probes recognizing at least 50% of the following biomarkers: ZNF329, LOC641518, TAP1, GBP2, RAB3IP, MYOF, MRPS10, ARF3, CLORF85, KCNE1L, BIN2, CACHD1, CYB5R3, FKBP12-EXI, CHM, DUS4L, STX5, AK3, BU580973, TCRA, CR608770, SPI1, SC65, FUNDC2, NDRG2, RPL28, SRP54, LOC643466, ZDHHC11B, NSUN5B, NDRG3, DHRS3, CPEB2, RAB3IP, PPID, FOXP1, EFNA1, C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, MAP4K5, CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, NSUN5, GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, EFNA1, GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1 biomarkers, contacting the biological sample from the subject with said collection of probes under conditions effective to permit hybridization of said probes to complementary nucleic acid molecules, if present, in the sample, detecting any hybridization as a result of said contacting, and identifying whether the subject has an autism spectrum disorder based on said detecting.

A final aspect of the present invention relates to a method of diagnosing whether a subject has a predisposition for developing an autism spectrum disorder involving obtaining a biological sample from a subject potentially having a predisposition for developing an autism spectrum disorder, providing one or more probes recognizing at least 50% of the following biomarkers: ZNF329, LOC641518, TAP1, GBP2, RAB3IP, MYOF, MRPS10, ARF3, CLORF85, KCNE1L, BIN2, CACHD1, CYB5R3, FKBP12-EXI, CHM, DUS4L, STX5, AK3, BU580973, TCRA, CR608770, SPI1, SC65, FUNDC2, NDRG2, RPL28, SRP54, LOC643466, ZDHHC11B, NSUN5B, NDRG3, DHRS3, CPEB2, RAB3IP, PPID, FOXP1, EFNA1, C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, MAP4K5, CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, NSUN5, GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, EFNA1, GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1 biomarkers, contacting the biological sample from the subject with said collection of probes under conditions effective to permit hybridization of said probes to complementary nucleic acid molecules, if present, in the sample, detecting any hybridization as a result of said contacting, and identifying whether the subject has a predisposition for developing an autism spectrum disorder based on said detecting.

The subject can be any mammal (e.g., mouse, rat, rabbit, hamster, guinea pig, cat, dog, pig, goat, cow, horse, primate, or human). Preferably, the subject is a human. More preferably, the subject is between 11 months to 4 years of age.

The initial determination that a subject is suspected of having an ASD, and, therefore, should be tested in accordance with the method of the present invention, can be made based on a general population-based screening method called the One-Year Well-Baby Check-Up Approach (Pierce et al., “Detecting, Studying, and Treating Autism Early: The One-year Well-Baby Check-Up Approach,” J Pediatr 159:458-465 (2011), which is hereby incorporated by reference in its entirety). For example, subject's failing the Communication and Symbolic Behavior Scales Development Profile (CSBS DP) can be suspected to have an ASD. In addition, failure of the Autism Diagnostic Observation Schedule (ADOS) and the clinical judgment of a Ph.D.-level psychologist can result in the determination that a subject is suspected of having an ASD.

The isolation of biological samples from a subject which contain nucleic acids is well known in the art. The biological sample can be sputum, blood, a blood fraction, tissue or fine needle biopsy sample, urine, stool, peritoneal fluid, or pleural fluid. Preferably, the biological sample is blood. In a more preferred embodiment, the biological sample is isolated peripheral blood mononuclear cells (PBMCs).

Methods for isolation of PBMCs are well known in the art. Typically, blood is collected from subjects into heparinized blood collection tubes by personnel trained in phlebotomy using sterile technique. The collected blood samples can be divided into aliquots and centrifuged, and the thin layer of cells between the erythrocyte layer and the plasma layer, which contains the PBMCs, is removed and used for analysis.

For the purposes of the present invention, the isolation of nucleic acids from the biological sample is desirable. Methods of isolating RNA and DNA from biological samples for use in the methods of the present invention are readily known in the art. These methods are described in detail in LABORATORY TECHNIQUES IN BIOCHEMISTRY AND MOLECULAR BIOLOGY: HYBRIDIZATION WITH NUCLEIC ACID PROBES, PART I. THEORY AND NUCLEIC ACID PREPARATION (P. Tijssen ed., Elsevier 1993), which is hereby incorporated by reference in its entirety. Total RNA can be isolated from a given sample using, for example, an acid guanidinium-phenol-chloroform extraction, a guanidinium isothiocyanate-ultracentrifugation method, or lithium chloride-SDS-urea method. PolyA⁺ mRNA can be isolated using oligo(dT) column chromatography or (dT)n magnetic beads (See e.g., SAMBROOK AND RUSSELL, MOLECULAR CLONING: A LABORATORY MANUAL (Cold Springs Laboratory Press, 1989) or CURRENT PROTOCOLS IN MOLECULAR BIOLOGY (Fred M. Ausubel et al. eds., 1992) which are hereby incorporated by reference in their entirety). See also WO/2000024939 to Dong et al., which is hereby incorporated by reference in its entirety, for complexity management and other nucleic acid sample preparation techniques.

It may be desirable to amplify the nucleic acid sample prior to detecting biomarker expression. One of skill in the art will appreciate that a method which maintains or controls for the relative frequencies of the amplified nucleic acids to achieve quantitative amplification should be used.

Typically, methods for amplifying nucleic acids employ a polymerase chain reaction (PCR) (See e.g., PCR TECHNOLOGY: PRINCIPLES AND APPLICATIONS FOR DNA AMPLIFICATION (Henry Erlich ed., Freeman Press 1992); PCR PROTOCOLS: A GUIDE TO METHODS AND APPLICATIONS (Michael Innis ed., Academic Press 1990); Mattila et al., “Fidelity of DNA Synthesis by the Thermococcus litoralis DNA Polymerase—An Extremely Heat Stable Enzyme with Proofreading Activity,” Nucleic Acids Res. 19:4967-73 (1991); Eckert et al., “DNA Polymerase Fidelity and the Polymerase Chain Reaction,” PCR Methods and Applications 1:17-24 (1991); and U.S. Pat. Nos. 4,683,202, 4,683,195, 4,800,159, 4,965,188, and 5,333,675 all to Mullis et al., which are hereby incorporated by reference in their entireties for all purposes). The sample can also be amplified on an array as described in U.S. Pat. No. 6,300,070 to Boles, which is hereby incorporated by reference in its entirety.

Other suitable amplification methods include the ligase chain reaction (LCR) (e.g. Wu et al., “The Ligation Amplification Reaction (LAR)—Amplification of Specific DNA Sequences Using Sequential Rounds of Template-Dependent Ligation,” Genomics 4:560-9 (1989), Landegren et al., “A Ligase-Mediated Gene Detection Technique,” Science 241:1077-80 (1988), and Barringer et al., “Blunt-End and Single-Strand Ligations by Escherichia coli Ligase: Influence on an In Vitro Amplification Scheme,” Gene 89:117-22 (1990), which are hereby incorporated by reference in their entirety); transcription amplification (Kwoh et al., “Transcription-Based Amplification System and Detection of Amplified Human Immunodeficiency Virus Type I with a Bead-Based Sandwich Hybridization Format,” Proc. Natl. Acad. Sci. USA 86:1173-7 (1989) and WO 88/10315 to Gingeras, which are hereby incorporated by reference in their entirety); self-sustained sequence replication (Guatelli et al., “Isothermal, In Vitro Amplification of Nucleic Acids by a Multienzyme Reaction Modeled After Retroviral Replication,” Proc. Natl. Acad. Sci. USA 87:1874-8 (1990) and WO 90/06995 to Gingeras, which are hereby incorporated by reference in their entirety); selective amplification of target polynucleotide sequences (U.S. Pat. No. 6,410,276 to Burg at al., which is hereby incorporated by reference in its entirety); consensus sequence primed polymerase chain reaction (CP-PCR) (U.S. Pat. No. 5,437,975 to McClelland, which is hereby incorporated by reference in its entirety); arbitrarily primed polymerase chain reaction (AP-PCR) (U.S. Pat. No. 5,413,909 to Bassam, and U.S. Pat. No. 5,861,245 to McClelland which are hereby incorporated by reference in their entirety); and nucleic acid based sequence amplification (NABSA) (See U.S. Pat. Nos. 5,409,818, 5,554,517, and 6,063,603 all to Davey, which are hereby incorporated by reference in their entirety). Other amplification methods that may be used are described in U.S. Pat. No. 5,242,794 to Whiteley; U.S. Pat. No. 5,494,810 to Barmy; and U.S. Pat. No. 4,988,617 to Landgren, which are hereby incorporated by reference in their entirety.

The biological sample is then contacted with said collection of probes under conditions effective to permit hybridization of said probes to complementary nucleic acid molecules, if present, in the sample. The probes comprise nucleotide sequences that are complementary to at least a region of mRNA or corresponding cDNA of the biomarkers listed above. As used herein, the term “hybridization” refers to the complementary base-pairing interaction of one nucleic acid with another nucleic acid that results in formation of a duplex, triplex, or other higher-ordered structure. Typically, the primary interaction is base specific, e.g., A/T and G/C, by Watson/Crick and Hoogsteen-type hydrogen bonding. Base-stacking and hydrophobic interactions can also contribute to duplex stability. Conditions for hybridizing detector probes to complementary and substantially complementary target sequences are well known in the art (see e.g., NUCLEIC ACID HYBRIDIZATION, A PRACTICAL APPROACH, B. Hames and S. Higgins, eds., IRL Press, Washington, D.C. (1985), which is hereby incorporated by reference in its entirety). In general, hybridization is influenced by, among other things, the length of the polynucleotides and their complements, the pH, the temperature, the presence of mono- and divalent cations, the proportion of G and C nucleotides in the hybridizing region, the viscosity of the medium, and the presence of denaturants. Such variables influence the time required for hybridization. Thus, the preferred hybridization conditions will depend upon the particular application. Such conditions, however, can be routinely determined by the person of ordinary skill in the art without undue experimentation. It will be appreciated that complementarity need not be perfect; there can be a small number of base pair mismatches that will minimally interfere with hybridization between the target sequence and single stranded nucleic acid probe. Thus, what is meant by complementarity herein is that the probes are sufficiently complementary to the target sequence to hybridize under the selected reaction conditions to achieve selective detection and measurement.

Detection of hybridization between said probes and corresponding target molecules from the biological sample can be performed by several assays known in the art that permit detection of the expression level of the biomarkers. As described herein, the “expression level” of a biomarker can be achieved by measuring any suitable value that is representative of the gene expression level. The measurement of gene expression levels can be direct or indirect. A direct measurement involves measuring the level or quantity of RNA or protein. An indirect measurement may involve measuring the level or quantity of cDNA, amplified RNA, DNA, or protein; the activity level of RNA or protein; or the level or activity of other molecules (e.g. a metabolite) that are indicative of the foregoing. The measurement of expression can be a measurement of the absolute quantity of a gene product. The measurement can also be a value representative of the absolute quantity, a normalized value (e.g., a quantity of gene product normalized against the quantity of a reference gene product), an averaged value (e.g., average quantity obtained at different time points or from different sample from a subject, or average quantity obtained using different probes, etc.), or a combination thereof.

In a preferred embodiment, hybridization is detected by measuring RNA expression level of the biomarkers. Measuring gene expression by quantifying mRNA expression can be achieved using any commonly used method known in the art including northern blotting and in situ hybridization (Parker et al., “mRNA: Detection by in Situ and Northern Hybridization,” Methods in Molecular Biology 106:247-283 (1999), which is hereby incorporated by reference in its entirety); RNAse protection assay (Hod et al., “A Simplified Ribonuclease Protection Assay,” Biotechniques 13:852-854 (1992), which is hereby incorporated by reference in its entirety); reverse transcription polymerase chain reaction (RT-PCR) (Weis et al., “Detection of Rare mRNAs via Quantitative RT-PCR,” Trends in Genetics 8:263-264 (1992), which is hereby incorporated by reference in its entirety); and serial analysis of gene expression (SAGE) (Velculescu et al., “Serial Analysis of Gene Expression,” Science 270:484-487 (1995); and Velculescu et al., “Characterization of the Yeast Transcriptome,” Cell 88:243-51 (1997), which is hereby incorporated by reference in its entirety).

In a more preferred embodiment, RNA expression level is measured using a nucleic acid hybridization assay or a nucleic acid amplification assay.

In a nucleic acid hybridization assay, the expression level of nucleic acids corresponding to biomarkers is detected using an array-based technique. These arrays, also commonly referred to as “microarrays” or “chips” have been generally described in the art, see e.g., U.S. Pat. No. 5,143,854 to Pirrung et al.; U.S. Pat. No. 5,445,934 to Fodor et al.; U.S. Pat. No. 5,744,305 to Fodor et al.; U.S. Pat. No. 5,677,195 to Winkler et al.; U.S. Pat. No. 6,040,193 to Winkler et al.; U.S. Pat. No. 5,424,186 to Fodor et al., which are all hereby incorporated by reference in their entirety. A microarray comprises an assembly of distinct polynucleotide or oligonucleotide probes immobilized at defined positions on a substrate. Arrays are formed on substrates fabricated with materials such as paper, glass, plastic (e.g., polypropylene, nylon), polyacrylamide, nitrocellulose, silicon, optical fiber or any other suitable solid or semi-solid support, and configured in a planar (e.g., glass plates, silicon chips) or three-dimensional (e.g., pins, fibers, beads, particles, microtiter wells, capillaries) configuration. Probes forming the arrays may be attached to the substrate by any number of ways including (i) in situ synthesis (e.g., high-density oligonucleotide arrays) using photolithographic techniques (see Fodor et al., “Light-Directed, Spatially Addressable Parallel Chemical Synthesis,” Science 251:767-773 (1991); Pease et al., “Light-Generated Oligonucleotide Arrays for Rapid DNA Sequence Analysis,” Proc. Natl. Acad. Sci. U.S.A. 91:5022-5026 (1994); Lockhart et al., “Expression Monitoring by Hybridization to High-Density Oligonucleotide Arrays,” Nature Biotechnology 14:1675 (1996); and U.S. Pat. No. 5,578,832 to Trulson; U.S. Pat. No. 5,556,752 to Lockhart; and U.S. Pat. No. 5,510,270 to Fodor, which are hereby incorporated by reference in their entirety); (ii) spotting/printing at medium to low-density (e.g., cDNA probes) on glass, nylon or nitrocellulose (Schena et al., “Quantitative Monitoring of Gene Expression Patterns with a Complementary DNA Microarray,” Science 270:467-470 (1995), DeRisi et al, “Use of a cDNA Microarray to Analyse Gene Expression Patterns in Human Cancer,” Nature Genetics 14:457-460 (1996); Shalon et al., “A DNA Microarray System for Analyzing Complex DNA Samples Using Two-Color Fluorescent Probe Hybridization,” Genome Res. 6:639-645 (1996); and Schena et al., “Proc. Natl. Acad. Sci. U.S.A. 93:10539-11286) (1995), which are hereby incorporated by reference in their entirety); (iii) masking (Maskos et al., “Oligonucleotide Hybridizations on Glass Supports: A Novel Linker for Oligonucleotide Synthesis and Hybridization Properties of Oligonucleotides Synthesised In Situ,” Nuc. Acids. Res. 20:1679-1684 (1992), which is hereby incorporated by reference in its entirety); and (iv) dot-blotting on a nylon or nitrocellulose hybridization membrane (see e.g., SAMBROOK AND RUSSELL, MOLECULAR CLONING: A LABORATORY MANUAL (Cold Springs Laboratory Press, 1989), which is hereby incorporated by reference in its entirety). Probes may also be noncovalently immobilized on the substrate by hybridization to anchors, by means of magnetic beads, or in a fluid phase such as in microtiter wells or capillaries. The probe molecules are generally nucleic acids such as DNA, RNA, PNA, and cDNA.

Fluorescently labeled cDNA for hybridization to the array may be generated through incorporation of fluorescent nucleotides by reverse transcription of RNA extracted from ASD subject samples of interest. Labeled cDNA applied to the array hybridizes with specificity to each nucleic acid probe spotted on the array. After stringent washing to remove non-specifically bound cDNA, the array is scanned by confocal laser microscopy or by another detection method, such as a CCD camera. Quantitation of hybridization of each arrayed element allows for assessment of corresponding mRNA abundance. With dual color fluorescence, separately labeled cDNA samples generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., “Parallel Human Genome Analysis: Microarray-Based Expression Monitoring of 1000 Genes,” “Proc. Natl. Acad. Sci. USA 93(20):10614-9 (1996), which is hereby incorporated by reference in its entirety).

In a preferred embodiment, in the nucleic acid hybridization assay, the expression levels of biomarkers informative of ASD diagnosis can be detected using the collections of probes of the present invention. In accordance with this aspect of the present invention, the nucleic acid probes of the present invention have a nucleotide sequence that is complementary to at least a portion of an RNA transcript or DNA nucleotide sequence encoded by a biomarker informative of ASD diagnosis.

A nucleic acid amplification assay that is a semi-quantitative or quantitative real-time polymerase chain reaction (RT-PCR) assay can also be performed. Because RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avian myeloblastosis virus reverse transcriptase (AMV-RT) and Moloney murine leukemia virus reverse transcriptase (MLV-RT), although others are also known and suitable for this purpose. The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. An exemplary PCR amplification system using Taq polymerase is TaqMan® PCR (Applied Biosystems, Foster City, Calif.). Taqman® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect the nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TagMan® RT-PCR can be performed using commercially available equipment, such as, for example, the ABI PRISM 7700® Sequence Detection System® (Perkin-Elmer-Applied Biosystems, Foster City, Calif., USA), or the Lightcycler (Roche Molecular Biochemicals, Mannheim, Germany).

In addition to the TagMan® primer/probe system, other quantitative methods and reagents for real-time PCR detection that are known in the art (e.g. SYBR green, Molecular Beacons, Scorpion Probes, etc.) are suitable for use in the methods of the present invention.

To minimize errors and the effect of sample-to-sample variation, RT-PCR is usually performed using an internal standard. The ideal internal standard is expressed at a constant level among different tissues, and is unaffected by the presence or absence of an ASD. RNAs most frequently used to normalize patterns of gene expression are mRNAs for the housekeeping genes glyceraldehyde-3-phosphate-dehydrogenase (GAPDH) and β-actin.

Real time PCR is compatible both with quantitative competitive PCR, where internal competitor for each target sequence is used for normalization and quantitative comparative PCR using a normalization gene contained within the sample, or a housekeeping gene for RT-PCR. For further details see, e.g., Heid et al., “Real Time Quantitative PCR,” Genome Research 6:986-994 (1996), which is incorporated by reference in its entirety.

The identification of whether or not the subject has an ASD based on said detecting can be performed by comparing the expression levels of said biomarkers with the expression levels of the same biomarkers in a typically developing subject. For the purposes of this invention, a “typically developing” subject is one who does not have or is not suspected of having any developmental disorder. Dependent upon the collection of probes used in the described method, comparisons may also be made to subjects with different disorders (e.g. Developmental Delay (DD) and Language Delay (LD)). The data generated from the detection of the previously biomarker expression levels can then be used to prepare a personalized genomic profile for a subject. The genomic profile can be used to establish a personalized treatment plan for the subject.

### EXAMPLES

The following examples are provided to illustrate embodiments of the present invention but are by no means intended to limit its scope.

### Materials and Methods for Examples 1-7

Protections.

The Institutional Review Boards of the University of California, San Diego and SUNY Upstate Medical University approved all procedures. All minor subjects assented to the study procedures, and one or both parents or legal guardians of each subject provided written informed consent for their child to participate.

Ascertainment.

Participants were obtained via a general population-based screening method called the One-Year Well-Baby Check-Up Approach, which engages community pediatricians to screen for autism during regular checkups (Pierce et al., “Detecting, Studying, and Treating Autism Early: The One-Year Well-Baby Check-up Approach,” J Pediatr (2011), which is hereby incorporated by reference in its entirety). All subjects failing the Communication and Symbolic Behavior Scales Developmental Profile (CSBS DP) Infant-Toddler questionnaire at their pediatrician's office were provisionally identified as at-risk for one of the disorders of interest and then referred for further evaluation. Subjects who passed the CSBS were identified as typically developing (TD) and some of these were also referred for further evaluation as unaffected comparison subjects. Using this approach, toddlers as young as 12-months were recruited and tracked every six months until at least their third birthday, thus allowing for the prospective study of autism beginning at 12 months.

Diagnoses.

ASDs were diagnosed based on failure of the Autism Diagnostic Observation Schedule (ADOS) (Lord et al., “Autism Diagnostic Observation Schedule (ADOS) Manual,” Western Psychological Services (2001), which is hereby incorporated by reference in its entirety) as well as the clinical judgment of a Ph.D.-level psychologist. While several ASD subjects were only one year old at the time of first blood sampling, all but one have been tracked and diagnosed with an ASD using the toddler module of the ADOS (Luyster et al., “The Autism Diagnostic Observation Schedule-Toddler Module: A New Module of a Standardized Diagnostic Measure for Austism Spectrum Disorders,” J Autism Dev Disord 39:1305-20 (2009), which is hereby incorporated by reference in its entirety) at age two or beyond, when the diagnosis of autism can be made reliably. Final diagnoses for participants with an ASD older than 30 months (including some of the youngest subjects who had previously been diagnosed with or at-risk for an ASD based on the ADOS) were confirmed with the Autism Diagnostic Interview-Revised (ADI-R) (Luyster et al., “The Autism Diagnostic Observation Schedule-Toddler Module: A New Module of a Standardized Diagnostic Measure for Austism Spectrum Disorders,” J Autism Dev Disord 39:1305-20 (2009), which is hereby incorporated by reference in its entirety). Subjects who failed the CSBS DP Infant-Toddler questionnaire but who passed the ADOS were diagnosed with language delay (LD) if one or both of the language subtest scores of the Mullen Scales of Early Learning were more than one standard deviation lower than expected for that age (T-score <40). Since a significant percentage of individuals with ASDs exhibit language deficits not restricted to the pragmatic component of language (Tager-Flusberg et al., “Present and Future Possibilities for Defining a Phenotype for Specific Language Impairment,” J Speech Lang Hear Res. 42:1275-8 (1999); Rapin et al., “Update on the Language Disorders of Individuals on the Autistic Spectrum,” Brain Dev. 25:166-72 (2003); Tager-Flusberg et al., “Identifying Neurocognitive Phenotypes in Autism,” Philos Trans R Soc Lond B Biol Sci 358:303-14 (2003); Tager-Flusberg et al., “Model Syndromes for Investigating Social Cognitive and Affective Neuroscience: A Comparison of Autism and Williams Syndrome,” Soc Cogn Affect Neurosci 1:175-82 (2006), each of which is hereby incorporated by reference in its entirety), this approach to diagnosing LD may not be highly specific; however, this criterion paired with ADOS evaluations not consistent with an ASD is sufficient to establish that these subjects have some language delay and are not on the autistic spectrum, making them suitable as an affected, non-autistic comparison sample for establishing the specificity of our ASD biomarkers. Subjects who failed the CSBS DP Infant-Toddler questionnaire but who passed the ADOS were diagnosed with global developmental delay (DD) if scores were more than one standard deviation lower than expected on three or more subtests of the Mullen Scales and the overall developmental quotient was more than one standard deviation lower than expected (<85). Subjects who failed the CSBS DP Infant-Toddler questionnaire but who passed the ADOS and who did not deviate from norms on any of the Mullen Scales or the developmental quotient were judged to be developing typically and identified as TIEs.

Laboratory Methods.

From each subject, 4 ml of venous blood was collected into EDTA-coated collection tubes and immediately transferred to an RNase-free laboratory where all subsequent procedures took place, including RNA isolation and storage at −80° C. within six hours. Methods of mRNA extraction, stabilization, isolation, storage, quantitation, reverse transcription, and microarray hybridization, as well as methods of microarray scanning and data import, normalization, and transformation, were consistent with prior work (Glatt et al, “Comparative Gene Expression Analysis of Blood and Brain Provides Concurrent Validation of SELENBP1 Up-Regulation in Schizophrenia,” Proceedings of the National Academy of Sciences of the United States of America 102:15533-8 (2005), which is hereby incorporated by reference in its entirety).

mRNA Extraction, Stabilization, Isolation, and Storage.

Total mRNA was extracted, stabilized, isolated, and stored from each blood sample in the same manner as described previously (Glatt et al., “Alternatively Spliced Genes as Biomarkers for Schizophrenia, Bipolar Disorder and Psychosis: a Blood-Based Spliceome-Profiling Exploratory Study,” Curr Pharmacogenom Pers Med. 7:164-188 (2009); Glatt et al., “Comparative Gene Expression Analysis of Blood and Brain Provides Concurrent Validation of SELENBP1 Up-Regulation in Schizophrenia,” Proc Natl Acad Sci USA 102:15533-15538 (2005); Tsuang et al., “Assessing the Validity of Blood-Based Gene Expression Profiles for the Classification of Schizophrenia and Bipolar Disorder: A Preliminary Report,” Am J Med Genet B Neuropsychiatr Genet. 133B:1-5 (2005), each of which are hereby incorporated by reference in its entirety). Briefly, 5 mL from each blood sample was passed over a LeukoLOCK filter, which was flushed with phosphate-buffered saline solution (PBS) and then fully saturated with RNAlater (Gonzales et al., “Isolate RNA from White Blood Cells Captured by a Novel Filter System,” Ambion TechNotes 12:24-25 (2005), which is hereby incorporated by reference in its entirety). Each LeukoLOCK filter, containing bound, isolated, stabilized, and purified white blood cells, was sealed and stored in a sterile box at −20° C. Once all samples were collected, LeukoLOCK filters were processed in a batch by flushing the filter with TRI reagent to lyse the cells and isolate mRNA. Eluted mRNA samples were stored at −20° C. until transferred to Scripps Genomic Medicine for quality assurance and microarray hybridization.

mRNA Quantitation and Quality Control.

The concentration of mRNA in each sample was quantified by the absorption of ultraviolet light at 260 nm. The quantity of mRNA in each sample exceeded the minimally sufficient amount required for microarray hybridization. The purity of each mRNA sample was estimated by the 260:280 nm absorbance ratio, with an acceptable range designated a priori as 1.7-2.1.

Reverse Transcription, Microarray Hybridization, and Scanning.

The samples were assayed in a batch on Illumina WG-6 v3 arrays (Illumina, San Diego, Calif.) according to the manufacturer's Direct Hybridization Gene Expression protocol, using 500 ng of total RNA from each sample. Briefly, each 500 ng sample was suspended in a volume of 11 μL and then amplified using Ambion's Illumina TotalPrep-96 RNA Amplification kit. Amplification steps consisted of the following: reverse transcription to synthesize first-strand cDNA; second-strand cDNA synthesis; cDNA purification; in vitro transcription to synthesize biotin-labeled cRNA; and cRNA purification. cRNA was then normalized to a concentration of 150 ng/μL (in 10-μL volume) and run through the Illumina Gene Expression assay which includes a 16- to 20-hour overnight incubation at 58° C. and subsequent scanning of the expression beadchip on a Bead Array Reader.

Microarray Data Normalization and Transformation.

Partek Genomics Suite software, version 6.3 (Partek, St. Louis, Mo.), was used for all analytic procedures performed on microarray scan data. First, raw probe-intensity values were imported; then corrections for background signal were applied using the robust multi-array average (RMA) method (Irizarry et al., “Exploration, Normalization, and Summaries of High Density Oligonucleotide Array Probe Level Data,” Biostatistics 4:249-264 (2003), which is hereby incorporated by reference in its entirety). An additional correction was made for the GC content of each probe. Expression levels of each probe underwent log 2 transformation to yield distributions of data that more closely approximated normality.

Quality Control and Final Sample Constitution.

The quality of mRNA samples was quantified by the RNA Integrity Number (RIN) and, according to convention (Schroeder et al., “The RIN: an RNA Integrity Number for Assigning Integrity Values to RNA Measurements,” BMC Molecular Biology 7:3 (2006), which is hereby incorporated by reference in its entirety) values of 6.0 or greater were deemed acceptable; values observed in samples ranged from 9.0-10.0. A total of 383 samples selected for analysis in Wave I had acceptable levels of mRNA quantity, purity, and quality. Once microarray data were generated, the first three principal components of global gene expression patterns in each individual were visualized. Subjects whose values on each of these three principal components were beyond four standard deviations from the grand mean (n=5) were removed from further analyses. Additional subjects were removed due to low signal intensity as observed by box-plot and evidenced by an average signal more than two standard deviations below the mean. The remaining 383 quality-assured samples were from a child proband in one of the six diagnostic groups described in Table 1.

TABLE 1
Demographic and Clinical Characteristics of the Six Diagnostic Groups

| | Diagnostic Group | | | | | |
|---|---|---|---|---|---|---|
| | AD | PDD | DD | LD | TD | TIE |
| Sample size, n | 138 | 35 | 17 | 34 | 84 | 75 |
| Sex, male:female | 105:33 | 29:6 | 13:4 | 26:8 | 47:37 | 49:26 |
| Age, mo, range | 11-49 | 12-41 | 13-42 | 11-31 | 11-45 | 12-37 |
| Mean (SD) | 26.6 (8.6) | 27.9 (7.31) | 24.5 (8.5) | 16.8 (4.6) | 23.4 (10.1) | 16.1 (5.5) |

The majority of subjects in the full sample and within each diagnostic group was of Caucasian ancestry, with no other ancestral group (Asian, Pacific Islander, African-American, American Indian, or Mixed) individually comprising more than 10% of any diagnostic group, and there were no differences in overall ancestral composition between any of the diagnostic groups (Pearson χ²₍₂₅₎=21.516; p=0.664). To increase sample size and inferential power, and maintain consistency with the conceptualization of AD and PDD as disorders on the same spectrum, these samples were pooled together into one ASD group for all subsequent analyses; likewise, both typically developing groups (TD and TIE) were pooled into one unaffected control (CNT) group for all subsequent analyses.

Microarray Data Analyses.

The ASD sample was split into two independent subgroups: a discovery sample and a replication sample. The two subsamples were drawn to be equivalent in sample size, age, sex, and diagnostic composition (AD versus PDD). The CNT sample was similarly split into discovery and replication samples drawn to be equivalent in size, age, sex, and diagnostic composition (TD versus TIE). The basic analytic model for identifying candidate biomarkers in the discovery samples was an analysis of covariance (ANCOVA), with each gene's expression intensity value as the dependent measure, diagnostic group (ASD versus CNT) and sex as fixed between-subjects factors, and age in months as a continuous covariate. Genes with expression levels influenced by at least a nominally significant main effect of diagnostic group (p<0.05) and with log 2 fold change in the ASD group of ≧|1.2| were identified as putative candidate ASD biomarkers and advanced to the next phase of analysis. These fairly liberal criteria were used to cast a wide net to catch all potentially informative genes, whereas false positive results would be pared off the putative biomarker set by subsequent model building, optimization, and replication steps. These genes were then used to build a support vector machine (SVM) classifier of the same discovery sample in which they were initially discovered. The SVM was configured as cost-based, with costs varied from 1 to 1001 in intervals of 100. The tolerance (termination criterion) of the SVM was set at 0.001. The kernel for the SVM was a radial basis function, with gamma equal to the inverse of the number of evaluated markers. The optimal (i.e., the most accurate and parsimonious) SVM was derived by shrinking centroids (which prunes highly correlated or redundant features) and 10-fold cross-validation. The optimal SVM classifier was then tested for classification accuracy in the fully independent ASD and CNT replication samples, and then re-evaluated in the presence of the LD and DD samples as affected but nonautistic comparators. The list of genes comprising the optimal classifier was subjected to the Database for Annotation, Visualization, and Integrated Discovery (DAVID) (Dennis et al., “DAVID: Database for Annotation, Visualization, and Integrated Discovery,” Genome Biol. 4:P3 (2003), which is hereby incorporated by reference in its entirety) algorithm to determine whether it was enriched with genes having particular functional, ontological, or structural annotations. Fold enrichments of such annotations were declared statistically significant only if they surpassed a Bonferroni-corrected p value threshold of α=0.05/number of terms evaluated in a particular category.

### Example 1

### Biomarker Panel 1

Among children identified with at least some developmental concerns, a biomarker panel is described that can differentiate children who go on to develop an ASD from children who go on to develop typically (TIE). This biomarker panel addresses the clinical question: “My 12-24 month-old child has developmental red flags. Is he more likely to develop an ASD or to develop typically?”

The optimal model for this classification problem is determined to comprise the expression levels of six genetic transcripts in a support vector machine of radial basis function with a cost of 601 and a gamma of 0.001. Expression levels of the genes in this classifier are measured in each individual, and these values are then combined in an equation (which was determined mathematically using a support vector machine-learning algorithm) that yields a dichotomous outcome (a 0 or 1) indicating whether the tested individual is more likely to have the disorder of interest or not. The methods of constructing and deploying support vector machine-learning algorithms on gene-expression data are described in detail by Byvatov and Schneider, “Support Vector Machine Applications in Bioinformatics,” Appl Bioinformatics 2(2):67-77 (2003), which is hereby incorporated by reference in its entirety. The six transcripts and their fold-change in ASD relative to typical development are as follows:

| | Fold-Change | |
|---|---|---|
| Probe | (ASD vs. TIE) | Gene Symbol |
| ILMN_1689059 | 1.24 | ZNF329 |
| ILMN_1707904 | 1.25 | LOC641518 |
| ILMN_1751079 | −1.43 | TAP1 |
| ILMN_1774077 | −1.35 | GBP2 |
| ILMN_1803197 | 1.14 | RAB3IP |
| ILMN_1810289 | −1.49 | MYOF |

The table below shows the historical performance of the optimal SVM classifier as determined in the Training Sample after 4-Fold Cross-Validation in 24 individuals with ASD vs 24 TIE individuals. The first lines of the tables below, as well as the following tables for Biomarker Panel 1, show the cross-tabulation of the number of individuals who were truly ASD and were called such by the SVM classifier (true-positive or TP calls), the number of individuals who were truly TIE and were called such by the classifier (true-negative or TN calls), the number of individuals who were truly ASD but were called TIE (false-negative or FN calls), and the number of individuals who were truly TIE but were called ASD (false-positive or FP calls). The subsequent lines of the table show the values of sensitivity (the percent of time the model finds true cases), specificity (the percent of time the model avoids calling unaffected individuals cases), positive predictive value (the percent of time those individuals called cases are actually cases) and negative predictive value (the percent of time those individuals called unaffected are actually unaffected), as well as the formulae used for their derivation based on TP, TN, FP, and FN. The final row of the table shows the area under the receiver operating characteristic curve, which is a measure of balance between sensitivity and specificity and can be interpreted as a measure of the model's accuracy.

| Real\Predicted | TIE | ASD |
|---|---|---|
| TIE | 22 | 2 |
| ASD | 6 | 18 |

| | |
|---|---|
| Sensitivity (TP/(TP + FN)): | 0.7500 |
| Specificity (TN/(FP + TN)): | 0.9167 |
| Positive Predictive Value (TP/(TP + FP)): | 0.9000 |
| Negative Predictive Value (TN/(FN + TN)): | 0.7857 |
| Area Under Curve (((TP/(TP + FN)) + (TN/FP + TN))*0.5): | 0.8333 |

The tables below show the results of test samples using Biomarker Panel 1 to differentiate between ASD and typical development.

Performance in Test Sample: 12 ASD vs 12 TIE, 12-24 mo. and individually
matched on sex and age within 1 month (excludes training samples)

| Real\Predicted | TIE | ASD |
|---|---|---|
| TIE | 8 | 4 |
| ASD | 1 | 11 |

| | |
|---|---|
| Sensitivity (TP/(TP + FN)): | 0.9167 |
| Specificity (TN/(FP + TN)): | 0.6667 |
| Positive Predictive Value (TP/(TP + FP)): | 0.7333 |
| Negative Predictive Value (TN/(FN + TN)): | 0.8889 |
| Area Under Curve (((TP/(TP + FN)) + (TN/FP + TN))*0.5): | 0.7917 |

Performance in Test Sample: All 41 ASD and 45 TIE, 12-24 mo.
(excludes training samples)

| Real\Predicted | TIE | ASD |
|---|---|---|
| TIE | 18 | 27 |
| ASD | 9 | 32 |

| | |
|---|---|
| Sensitivity (TP/(TP + FN)): | 0.7805 |
| Specificity (TN/(FP + TN)): | 0.4000 |
| Positive Predictive Value (TP/(TP + FP)): | 0.5424 |
| Negative Predictive Value (TN/(FN + TN)): | 0.6667 |
| Area Under Curve (((TP/(TP + FN)) + (TN/FP + TN))*0.5): | 0.5902 |

Performance in Test Sample: All 149 ASD vs 51
TIE (excludes training samples)

| Real\Predicted | TIE | MD |
|---|---|---|
| TIE | 20 | 31 |
| ASD | 45 | 104 |

| | |
|---|---|
| Sensitivity (TP/(TP + FN)): | 0.6980 |
| Specificity (TN/(FP + TN)): | 0.3922 |
| Positive Predictive Value (TP/(TP + FP)): | 0.7704 |
| Negative Predictive Value (TN/(FN + TN)): | 0.3077 |
| Area Under Curve (((TP/(TP + FN)) + (TN/FP + TN))*0.5): | 0.5451 |

According to conventions advocated by Hanley and McNeil (Hanley and McNeil, “The Meaning and Use of the Area Under a Receiver Operating Characteristic (ROC) Curve,” Radiology 143(1):29-36 (1982), which is hereby incorporated by reference in its entirety, diagnostic accuracy may be considered outstanding, excellent, good, fair, or poor if AUC is in the range of 0.9-1.0, 0.81-0.90, 0.71-0.80, 0.61-0.70, or <0.60, respectively.

### Example 2

### Biomarker Panel 2

Among children identified with at least some developmental concerns, a biomarker panel is described that can differentiate children who go on to develop an ASD from children who go on to develop a DD or LD. This biomarker panel addresses the clinical question: “My 12-24 month-old child has developmental red flags. Is he more likely to develop an ASD or to develop a DD or LD?”

The optimal model for this classification problem is determined to comprise the expression levels of 16 genetic transcripts in a support vector machine of radial basis function with a cost of 501 and a gamma of 0.001. Expression levels of the genes in this classifier are measured in each individual, and these values are then combined in an equation (which was determined mathematically using a support vector machine-learning algorithm) that yields a dichotomous outcome (a 0 or 1) indicating whether the tested individual is more likely to have the disorder of interest or not. The methods of constructing and deploying support vector machine-learning algorithms on gene-expression data are described in detail by Byvatov and Schneider, “Support Vector Machine Applications in Bioinformatics,” Appl Bioinformatics 2(2):67-77 (2003), which is hereby incorporated by reference in its entirety. The 16 transcripts and their fold-change in ASD relative to typical development are as follows:

| | Gene | Fold-Change |
|---|---|---|
| Probe | Symbol | (ASD vs. DD/LD) |
| ILMN_1663664 | MRPS10 | 1.17 |
| ILMN_1682938 | ARF3 | −1.19 |
| ILMN_1698243 | C1orf85 | −1.24 |
| ILMN_1711650 | KCNE1L | −1.1 |
| ILMN_1726342 | BIN2 | −1.25 |
| ILMN_1738854 | CACHD1 | 1.14 |
| ILMN_1740441 | CYB5R3 | −1.14 |
| ILMN_1757072 | FKBP12-Exi | −1.21 |
| ILMN_1771238 | CHM | 1.15 |
| ILMN_1776858 | DUS4L | 1.12 |
| ILMN_1777444 | STX5 | −1.11 |
| ILMN_1778173 | AK3 | 1.17 |
| ILMN_1832672 | BU580973 | 1.11 |
| ILMN_1843100 | TCRA | 1.12 |
| ILMN_1884750 | CR608770 | 1.15 |
| ILMN_2392043 | SPI1 | −1.34 |

The table below shows the historical performance of the optimal SVM classifier as determined in the Training Sample after 3-Fold Cross-Validation in 27 individuals with ASD vs 27 DD/LD individuals. The first lines of the table below, as well as the following tables for Biomarker Panel 2, show the cross-tabulation of the number of individuals who were truly ASD and were called such by the SVM classifier (true-positive or TP calls), the number of individuals who were truly DD/LD and were called such by the classifier (true-negative or TN calls), the number of individuals who were truly ASD but were called DD/LD (false-negative or FN calls), and the number of individuals who were truly DD/LD but were called ASD (false-positive or FP calls). The subsequent lines of the table show the values of sensitivity (the percent of time the model finds true cases), specificity (the percent of time the model avoids calling unaffected individuals cases), positive predictive value (the percent of time those individuals called cases are actually cases) and negative predictive value (the percent of time those individuals called unaffected are actually unaffected), as well as the formulae used for their derivation based on TP, TN, FP, and FN. The final row of the table shows the area under the receiver operating characteristic curve, which is a measure of balance between sensitivity and specificity and can be interpreted as a measure of the model's accuracy.

Performance in Training Sample: 3-Fold Cross-Validation of
27 ASD vs 27 DD/LD

| Real\Predicted | DD/LD | ASD |
|---|---|---|
| DD/LD | 21 | 6 |
| ASD | 3 | 24 |

| | |
|---|---|
| Sensitivity (TP/(TP + FN)): | 0.8889 |
| Specificity (TN/(FP + TN)): | 0.7778 |
| Positive Predictive Value (TP/(TP + FP)): | 0.8000 |
| Negative Predictive Value (TN/(FN + TN)): | 0.8750 |
| Area Under Curve (((TP/(TP + FN)) + | 0.8333 |
| (TN/FP + TN))*0.5): | |

The tables below show the results of test samples using Biomarker Panel 2 to differentiate between ASD and DD/LD.

Performance in Test Sample: 13 ASD vs 13 DD/LD, 12-24 mo. and
individually matched on sex and age within 1 month
(excludes training samples)

| Real\Predicted | DD/LD | ASD |
|---|---|---|
| DD/LD | 9 | 4 |
| ASD | 1 | 12 |

| | |
|---|---|
| Sensitivity (TP/(TP + FN)): | 0.9231 |
| Specificity (TN/(FP + TN)): | 0.6923 |
| Positive Predictive Value (TP/(TP + FP)): | 0.7500 |
| Negative Predictive Value (TN/(FN + TN)): | 0.9000 |
| Area Under Curve (((TP/(TP + FN)) + | 0.8077 |
| (TN/FP + TN))*0.5): | |

Performance in Test Sample: All 38 ASD vs 14 DD/LD, 12-24 mo.
(excludes training samples)

| Real\Predicted | DD/LD | ASD |
|---|---|---|
| DD/LD | 9 | 5 |
| ASD | 7 | 31 |

| | |
|---|---|
| Sensitivity (TP/(TP + FN)): | 0.8158 |
| Specificity (TN/(FP + TN)): | 0.6429 |
| Positive Predictive Value (TP/(TP + FP)): | 0.8611 |
| Negative Predictive Value (TN/(FN + TN)): | 0.5625 |
| Area Under Curve (((TP/(TP + FN)) + | 0.7293 |
| (TN/FP + TN))*0.5): | |

Performance in Test Sample: All 146 ASD vs 24 DD/LD
(excludes training samples)

| Real\Predicted | DD/LD | ASD |
|---|---|---|
| DD/LD | 10 | 14 |
| ASD | 31 | 115 |

| | |
|---|---|
| Sensitivity (TP/(TP + FN)): | 0.7877 |
| Specificity (TN/(FP + TN)): | 0.4167 |
| Positive Predictive Value (TP/(TP + FP)): | 0.8915 |
| Negative Predictive Value (TN/(FN + TN)): | 0.2439 |
| Area Under Curve (((TP/(TP + FN)) + | 0.6022 |
| (TN/FP + TN))*0.5): | |

According to conventions advocated by Hanley and McNeil (Hanley and McNeil, “The Meaning and Use of the Area Under a Receiver Operating Characteristic (ROC) Curve,” Radiology 143(1):29-36 (1982), which is hereby incorporated by reference in its entirety, diagnostic accuracy may be considered outstanding, excellent, good, fair, or poor if AUC is in the range of 0.9-1.0, 0.81-0.90, 0.71-0.80, 0.61-0.70, or <0.60, respectively.

### Example 3

### Biomarker Panel 3

Among children identified with at least some developmental concerns, a biomarker panel is described that can differentiate children who go on to develop an ASD, DD, or LD from children who go on to develop typically. This biomarker panel addresses the clinical question: “My 12-24 month-old child has developmental red flags. Is he more likely to develop an ASD, DD, or LD or to develop typically?”

The optimal model for this classification problem is determined to comprise the expression levels of 16 genetic transcripts in a support vector machine of radial basis function with a cost of 101 and a gamma of 0.001. Expression levels of the genes in this classifier are measured in each individual, and these values are then combined in an equation (which was determined mathematically using a support vector machine-learning algorithm) that yields a dichotomous outcome (a 0 or 1) indicating whether the tested individual is more likely to have the disorder of interest or not. The methods of constructing and deploying support vector machine-learning algorithms on gene-expression data are described in detail by Byvatov and Schneider, “Support Vector Machine Applications in Bioinformatics,” Appl Bioinformatics 2(2):67-77 (2003), which is hereby incorporated by reference in its entirety. The 16 transcripts and their fold-change in ASD relative to typical development are as follows:

| | Gene | Fold-Change |
|---|---|---|
| Probe | Symbol | (ASD/DD/LD vs. TIE) |
| ILMN_1655663 | SC65 | 1.09 |
| ILMN_1669118 | FUNDC2 | 1.07 |
| ILMN_1670535 | NDRG2 | 1.1 |
| ILMN_1673509 | RPL28 | 1.19 |
| ILMN_1685173 | SRP54 | −1.1 |
| ILMN_1702519 | LOC643466 | 1.13 |
| ILMN_1713553 | ZDHHC11B | −1.05 |
| ILMN_1722450 | NSUN5B | 1.11 |
| ILMN_1738229 | NDRG3 | 1.15 |
| ILMN_1752478 | DHRS3 | 1.18 |
| ILMN_1774948 | CPEB2 | 1.07 |
| ILMN_1803197 | RAB3IP | 1.1 |
| ILMN_2190851 | PPID | −1.13 |
| ILMN_2250923 | FOXP1 | 1.12 |
| ILMN_2371053 | EFNA1 | 1.08 |
| ILMN_2371055 | EFNA1 | 1.17 |

The table below shows the historical performance of the optimal SVM classifier as determined in the Training Sample after 4-Fold Cross-Validation in 39 individuals with ASD/DD/LD vs 39 TIE individuals. The first lines of the table below, as well as the following tables for Biomarker Panel 3, show the cross-tabulation of the number of individuals who were truly ASD/DD/LD and were called such by the SVM classifier (true-positive or TP calls), the number of individuals who were truly TIE and were called such by the classifier (true-negative or TN calls), the number of individuals who were truly ASD/DD/LD but were called TIE (false-negative or FN calls), and the number of individuals who were truly TIE but were called ASD/DD/LD (false-positive or FP calls). The subsequent lines of the table show the values of sensitivity (the percent of time the model finds true cases), specificity (the percent of time the model avoids calling unaffected individuals cases), positive predictive value (the percent of time those individuals called cases are actually cases) and negative predictive value (the percent of time those individuals called unaffected are actually unaffected), as well as the formulae used for their derivation based on TP, TN, FP, and FN. The final row of the table shows the area under the receiver operating characteristic curve, which is a measure of balance between sensitivity and specificity and can be interpreted as a measure of the model's accuracy.

Performance in Training Sample: Four-Fold Cross-Validation of 39
ASD/DD/LD vs 39 TIE

| | | ASD/ |
|---|---|---|
| Real\Predicted | TIE | DD/LD |
| TIE | 34 | 5 |
| ASD/DD/LD | 6 | 33 |

| | |
|---|---|
| Sensitivity (TP/(TP + FN)): | 0.8718 |
| Specificity (TN/(FP + TN)): | 0.8462 |
| Positive Predictive Value (TP/(TP + FP)): | 0.8500 |
| Negative Predictive Value (TN/(FN + TN)): | 0.8684 |
| Area Under Curve (((TP/(TP + FN)) + | 0.8590 |
| (TN/FP + TN))*0.5): | |

The tables below show the results of test samples using Biomarker Panel 3 to differentiate between ASD and DD/LD as a group and typical development.

Performance in Test Sample: 19 ASD/DD/LD vs 19 TIE, 12-24 mo. and
individually matched on sex and age within 1 month
(excludes training samples)

| | | ASD/ |
|---|---|---|
| Real\Predicted | TIE | DD/LD |
| TIE | 15 | 4 |
| ASD/DD/LD | 2 | 17 |

| | |
|---|---|
| Sensitivity (TP/(TP + FN)): | 0.7895 |
| Specificity (TN/(FP + TN)): | 0.8947 |
| Positive Predictive Value (TP/(TP + FP)): | 0.8824 |
| Negative Predictive Value (TN/(FN + TN)): | 0.8095 |
| Area Under Curve (((TP/(TP + FN)) + | 0.8421 |
| (TN/FP + TN))*0.5): | |

Performance in Test Sample: All ASD/DD/LD and TIE, 12-24 mo. and
individually matched on sex and age within 1 month
(excludes training samples)

| | | ASD/ |
|---|---|---|
| Real\Predicted | TIE | DD/LD |
| TIE | 41 | 17 |
| ASD/DD/LD | 10 | 48 |

| | |
|---|---|
| Sensitivity (TP/(TP + FN)): | 0.7069 |
| Specificity (TN/(FP + TN)): | 0.8276 |
| Positive Predictive Value (TP/(TP + FP)): | 0.8039 |
| Negative Predictive Value (TN/(FN + TN)): | 0.7385 |
| Area Under Curve (((TP/(TP + FN)) + | 0.7672 |
| (TN/FP + TN))*0.5): | |

Performance in Test Sample: All ASD/DD/LD vs TIE
(excludes training samples)

| | | ASD/ |
|---|---|---|
| Real\Predicted | TIE | DD/LD |
| TIE | 65 | 41 |
| ASD/DD/LD | 13 | 56 |

| | |
|---|---|
| Sensitivity (TP/(TP + FN)): | 0.6132 |
| Specificity (TN/(FP + TN)): | 0.8116 |
| Positive Predictive Value (TP/(TP + FP)): | 0.8333 |
| Negative Predictive Value (TN/(FN + TN)): | 0.5773 |
| Area Under Curve (((TP/(TP + FN)) + | 0.7124 |
| (TN/FP + TN))*0.5): | |

According to conventions advocated by Hanley and McNeil (Hanley and McNeil, “The Meaning and Use of the Area Under a Receiver Operating Characteristic (ROC) Curve,” Radiology 143(1):29-36 (1982), which is hereby incorporated by reference in its entirety, diagnostic accuracy may be considered outstanding, excellent, good, fair, or poor if AUC is in the range of 0.9-1.0, 0.81-0.90, 0.71-0.80, 0.61-0.70, or <0.60, respectively.

### Example 4

### Biomarker Panel 4

Among children identified with at least some developmental concerns, a biomarker panel is described that can differentiate children who go on to develop an ASD from children who go on to develop a DD or LD and from children who go on to develop typically. This biomarker panel addresses the clinical question: “My 12-24 month-old child has developmental red flags. Is he more likely to develop an ASD, to develop a DD or LD, or to develop typically?”

The optimal model for this classification problem is determined to comprise the expression levels of 36 genetic transcripts in a support vector machine of radial basis function with a cost of 101 and a gamma of 0.01. Expression levels of the genes in this classifier are measured in each individual, and these values are then combined in an equation (which was determined mathematically using a support vector machine-learning algorithm) that yields a dichotomous outcome (a 0 or 1) indicating whether the tested individual is more likely to have the disorder of interest or not. The methods of constructing and deploying support vector machine-learning algorithms on gene-expression data are described in detail by Byvatov and Schneider, “Support Vector Machine Applications in Bioinformatics,” Appl Bioinformatics 2(2):67-77 (2003), which is hereby incorporated by reference in its entirety. The 36 transcripts and their fold-change in ASD relative to typical development are as follows:

| | | Fold- | Fold- | Fold- |
|---|---|---|---|---|
| | | Change | Change | Change |
| | Gene | (ASD vs. | (DD/LD vs. | (ASD vs. |
| Probe | Symbol | TIE) | TIE) | DD/LD) |
| ILMN_1658439 | C5orf44 | −1.08 | −1.33 | 1.23 |
| ILMN_1658853 | ARHGAP25 | −1.32 | −1.29 | −1.03 |
| ILMN_1665655 | CTDSPL2 | −1.1 | −1.24 | 1.12 |
| ILMN_1674411 | CKAP2 | −1.15 | −1.29 | 1.13 |
| ILMN_1677997 | MAZ | −1.26 | −1.04 | −1.22 |
| ILMN_1684042 | BET1 | −1.07 | −1.27 | 1.19 |
| ILMN_1685173 | SRP54 | −1.1 | −1.11 | 1.01 |
| ILMN_1689327 | CR617556 | 1.03 | −1.15 | 1.19 |
| ILMN_1699476 | RPE | −1.14 | −1.38 | 1.21 |
| ILMN_1701507 | EHHADH | −1.07 | −1.14 | 1.06 |
| ILMN_1704084 | CMAH | −1.15 | −1.33 | 1.16 |
| ILMN_1714850 | ECD | −1.07 | −1.24 | 1.16 |
| ILMN_1727348 | NMD3 | −1.11 | −1.3 | 1.17 |
| ILMN_1732489 | SLC10A7 | −1.06 | −1.27 | 1.19 |
| ILMN_1738736 | SNX4 | −1.07 | −1.29 | 1.21 |
| ILMN_1743208 | NEDD1 | −1.1 | −1.33 | 1.21 |
| ILMN_1750029 | GABPA | −1.13 | −1.55 | 1.37 |
| ILMN_1763884 | Magmas | 1.17 | 1.29 | −1.1 |
| ILMN_1770515 | UBE2V2 | −1.06 | −1.34 | 1.26 |
| ILMN_1774077 | GBP2 | −1.4 | −1.26 | −1.11 |
| ILMN_1795524 | C15orf44 | −1.07 | −1.18 | 1.11 |
| ILMN_1810181 | PCGF6 | −1.03 | −1.22 | 1.18 |
| ILMN_1814044 | CABIN1 | 1.34 | 1.59 | −1.19 |
| ILMN_1815345 | EIF3J | −1.06 | −1.28 | 1.21 |
| ILMN_1818632 | Hs.561844 | 1.39 | 1.2 | 1.16 |
| ILMN_2043845 | IMPACT | −1.06 | −1.13 | 1.07 |
| ILMN_2048700 | ATAD2 | −1.12 | −1.24 | 1.11 |
| ILMN_2124386 | RGL2 | 1.16 | 1.42 | −1.22 |
| ILMN_2139035 | CASD1 | −1.1 | −1.36 | 1.24 |
| ILMN_2140389 | TMEM185A | 1.17 | 1.27 | −1.08 |
| ILMN_2212878 | ESM1 | −1.08 | −1.13 | 1.05 |
| ILMN_2240009 | ADSSL1 | −1.09 | −1.14 | 1.05 |
| ILMN_2370882 | ACSL5 | −1.15 | −1.22 | 1.06 |
| ILMN_2371470 | C1orf124 | −1.12 | −1.24 | 1.11 |
| ILMN_2378376 | CYB561 | −1.22 | −1.23 | 1.01 |
| ILMN_2408908 | MAP4K5 | −1.06 | −1.26 | 1.19 |

The table below shows the historical performance of the optimal SVM classifier as determined in the Training Sample after 3-Fold Cross-Validation in 21 individuals with ASD vs 21 DD/LD vs 21 TIE individuals. The first lines of the table below, as well as the following tables for Biomarker Panel 4, show the cross-tabulation of the number of individuals who were truly DD/LD, or ASD and were called such by the SVM classifier (true-positive or TP calls), the number of individuals who were truly TIE and were called such by the classifier (true-negative or TN calls), the number of individuals who were truly DD/LD, or ASD but were called TIE (false-negative or FN calls), and the number of individuals who were truly TIE but were called DD/LD or ASD (false-positive or FP calls). The subsequent lines of the table show the values of sensitivity (the percent of time the model finds true cases), specificity (the percent of time the model avoids calling unaffected individuals cases), positive predictive value (the percent of time those individuals called cases are actually cases) and negative predictive value (the percent of time those individuals called unaffected are actually unaffected), as well as the formulae used for their derivation based on TP, TN, FP, and FN. The final row of the table shows the area under the receiver operating characteristic curve, which is a measure of balance between sensitivity and specificity and can be interpreted as a measure of the model's accuracy.

Performance in Training Sample: 3-Fold Cross-Validation in 21 ASD
vs 21 DD/LD vs 21 TIE

| Real\Predicted | TIE | DD/LD | ASD |
|---|---|---|---|
| TIE | 17 | 2 | 12 |
| DD/LD | 2 | 13 | 16 |
| ASD | 2 | 4 | 15 |

| | |
|---|---|
| Sensitivity (TP/(TP + FN)): | 0.7143 |
| Specificity (TN/(FP + TN)): | 0.8095 |
| Positive Predictive Value (TP/(TP + FP)): | 0.6522 |
| Negative Predictive Value (TN/(FN + TN)): | 0.8500 |
| Area Under Curve (((TP/(TP + FN)) + | 0.7619 |
| (TN/FP + TN))*0.5): | |

The tables below show the results of test samples using Biomarker Panel 4 to differentiate between ASD, DD/LD, and typical development.

Performance in Test Sample: 10 ASD vs 10 DD/LD vs 10 TIE,
12-24 mo. and individually matched on sex and age within 1 mo.
(excludes training samples)

| Real\Predicted | TIE | DD/LD | ASD |
|---|---|---|---|
| TIE | 6 | 0 | 4 |
| DD/LD | 0 | 4 | 6 |
| ASD | 0 | 1 | 9 |

| | |
|---|---|
| Sensitivity (TP/(TP + FN)): | 0.9000 |
| Specificity (TN/(FP + TN)): | 0.5000 |
| Positive Predictive Value (TP/(TP + FP)): | 0.4737 |
| Negative Predictive Value (TN/(FN + TN)): | 0.9091 |
| Area Under Curve (((TP/(TP + FN)) + | 0.7000 |
| (TN/FP + TN))*0.5): | |

Performance in Test Sample: All 44 ASD vs 20 DD/LD vs 48 TIE,
12-24 mo. (excludes training samples)

| Real\Predicted | TIE | DD/LD | ASD |
|---|---|---|---|
| TIE | 15 | 3 | 30 |
| DD/LD | 0 | 4 | 16 |
| ASD | 8 | 4 | 32 |

| | |
|---|---|
| Sensitivity (TP/(TP + FN)): | 0.7273 |
| Specificity (TN/(FP + TN)): | 0.3235 |
| Positive Predictive Value (TP/(TP + FP)): | 0.4103 |
| Negative Predictive Value (TN/(FN + TN)): | 0.6471 |
| Area Under Curve (((TP/(TP + FN)) + | 0.5254 |
| (TN/FP + TN))*0.5): | |

Performance in Test Sample: All 152 ASD vs 30 DD/LD vs TIE
(excludes training samples)

| Real\Predicted | TIE | DD/LD | ASD |
|---|---|---|---|
| TIE | 15 | 3 | 36 |
| DD/LD | 2 | 5 | 23 |
| ASD | 22 | 13 | 117 |

| | |
|---|---|
| Sensitivity (TP/(TP + FN)): | 0.7697 |
| Specificity (TN/(FP + TN)): | 0.2976 |
| Positive Predictive Value (TP/(TP + FP)): | 0.6648 |
| Negative Predictive Value (TN/(FN + TN)): | 0.4167 |
| Area Under Curve (((TP/(TP + FN)) + | 0.5337 |
| (TN/FP + TN))*0.5): | |

According to conventions advocated by Hanley and McNeil (Hanley and McNeil, “The Meaning and Use of the Area Under a Receiver Operating Characteristic (ROC) Curve,” Radiology 143(1):29-36 (1982), which is hereby incorporated by reference in its entirety, diagnostic accuracy may be considered outstanding, excellent, good, fair, or poor if AUC is in the range of 0.9-1.0, 0.81-0.90, 0.71-0.80, 0.61-0.70, or <0.60, respectively.

### Example 5

### Biomarker Panel 5

Among all children, a biomarker panel is described that can differentiate children who go on to develop an ASD from children who go on to develop typically (TD/TIE). This biomarker panel addresses the clinical question: “Is my child more likely to develop an ASD or to develop typically?”

The optimal model for this classification problem is determined to comprise the expression levels of 36 genetic transcripts in a support vector machine of radial basis function with a cost of 701 and a gamma of 0.001. Expression levels of the genes in this classifier are measured in each individual, and these values are then combined in an equation (which was determined mathematically using a support vector machine-learning algorithm) that yields a dichotomous outcome (a 0 or 1) indicating whether the tested individual is more likely to have the disorder of interest or not. The methods of constructing and deploying support vector machine-learning algorithms on gene-expression data are described in detail by Byvatov and Schneider, “Support Vector Machine Applications in Bioinformatics,” Appl Bioinformatics 2(2):67-77 (2003), which is hereby incorporated by reference in its entirety. The 36 transcripts and their fold-change in ASD relative to typical development are as follows:

| | | Fold-Change |
|---|---|---|
| Illumina Probe | Gene Symbol | (ASD vs. TD/TIE) |
| ILMN_1656920 | CRIP1 | 1.22 |
| ILMN_1662243 | ING1 | 1.08 |
| ILMN_1668588 | LILRB1 | −1.16 |
| ILMN_1668984 | SPNS3 | 1.22 |
| ILMN_1672611 | CDH11 | 1.07 |
| ILMN_1674953 | LOC642403 | 1.07 |
| ILMN_1678454 | CASP4 | −1.22 |
| ILMN_1682781 | TEAD2 | 1.14 |
| ILMN_1691747 | KHDRBS3 | 1.14 |
| ILMN_1703558 | FHL3 | 1.26 |
| ILMN_1707904 | LOC641518 | 1.19 |
| ILMN_1711087 | EPPK1 | 1.13 |
| ILMN_1714433 | MARCKSL1 | 1.21 |
| ILMN_1716730 | FAM44B | 1.11 |
| ILMN_1722855 | VEGFB | 1.16 |
| ILMN_1740927 | LYRM4 | 1.14 |
| ILMN_1748976 | AB007962 | −1.08 |
| ILMN_1752032 | PPP2R3B | 1.13 |
| ILMN_1763516 | SPINK2 | 1.18 |
| ILMN_1769409 | C9orf123 | 1.16 |
| ILMN_1772058 | PANK2 | −1.06 |
| ILMN_1776993 | COG2 | 1.1 |
| ILMN_1796180 | CRY2 | 1.12 |
| ILMN_1800626 | SESN1 | 1.25 |
| ILMN_1815519 | EPN2 | 1.19 |
| ILMN_1844464 | IL23A | 1.19 |
| ILMN_1846630 | BE439556 | 1.06 |
| ILMN_1914495 | DB050967 | 1.06 |
| ILMN_2073012 | TMEM203 | 1.1 |
| ILMN_2148668 | RCBTB2 | −1.2 |
| ILMN_2197519 | ZNF627 | 1.09 |
| ILMN_2287911 | CMTM1 | −1.08 |
| ILMN_2317457 | HSD11B1L | 1.07 |
| ILMN_2320330 | MAL | 1.23 |
| ILMN_2405628 | TOP1MT | 1.18 |
| ILMN_2408400 | NSUN5 | 1.11 |

The table below shows the historical performance of the optimal SVM classifier as determined in the Training Sample after 5-Fold Cross-Validation in 35 individuals with ASD vs 35 TD/TIE individuals. The first lines of the table below, as well as the following tables for Biomarker Panel 5, show the cross-tabulation of the number of individuals who were truly ASD and were called such by the SVM classifier (true-positive or TP calls), the number of individuals who were truly TD/TIE and were called such by the classifier (true-negative or TN calls), the number of individuals who were truly ASD but were called TD/TIE (false-negative or FN calls), and the number of individuals who were truly TD/TIE but were called ASD (false-positive or FP calls). The subsequent lines of the table show the values of sensitivity (the percent of time the model finds true cases), specificity (the percent of time the model avoids calling unaffected individuals cases), positive predictive value (the percent of time those individuals called cases are actually cases) and negative predictive value (the percent of time those individuals called unaffected are actually unaffected), as well as the formulae used for their derivation based on TP, TN, FP, and FN. The final row of the table shows the area under the receiver operating characteristic curve, which is a measure of balance between sensitivity and specificity and can be interpreted as a measure of the model's accuracy.

Performance in Training Sample:
5-Fold Cross-Validation in 35 ASD vs 35 TD/TIE

| Real\Predicted | TD/TIE | ASD |
|---|---|---|
| TD/TIE | 29 | 6 |
| ASD | 2 | 33 |

| | |
|---|---|
| Sensitivity (TP/(TP + FN)): | 0.9429 |
| Specificity (TN/(FP + TN)): | 0.8286 |
| Positive Predictive Value (TP/(TP + FP)): | 0.8462 |
| Negative Predictive Value (TN/(FN + TN)): | 0.9355 |
| Area Under Curve (((TP/(TP + FN)) + (TN/FP + TN))*0.5): | 0.8857 |

The tables below show the results of test samples using Biomarker Panel 5 to differentiate between ASD and typical development in all children.

Performance in Test Sample: 17 ASD vs 17 TD/TIE,
12-24 mo. and individually matched on sex
and age within 1 month (excludes training samples)

| Real\Predicted | TD/TIE | ASD |
|---|---|---|
| TD/TIE | 14 | 3 |
| ASD | 3 | 14 |

| | |
|---|---|
| Sensitivity (TP/(TP + FN)): | 0.8235 |
| Specificity (TN/(FP + TN)): | 0.8235 |
| Positive Predictive Value (TP/(TP + FP)): | 0.8235 |
| Negative Predictive Value (TN/(FN + TN)): | 0.8235 |
| Area Under Curve (((TP/(TP + FN)) + (TN/FP + TN))*0.5): | 0.8235 |

Performance in Test Sample: All 30 ASD vs
85 TD/TIE, 12-24 mo. (excludes training samples)

| Real\Predicted | TD/TIE | VSD |
|---|---|---|
| TD/TIE | 48 | 137 |
| ASD | 5 | 25 |

| | |
|---|---|
| Sensitivity (TP/(TP + FN)): | 0.8333 |
| Specificity (TN/(FP + TN)): | 0.5647 |
| Positive Predictive Value (TP/(TP + FP)): | 0.4032 |
| Negative Predictive Value (TN/(FN + TN)): | 0.9057 |
| Area Under Curve (((TP/(TP + FN)) + (TN/FP + TN))*0.5): | 0.6990 |

Performance in Test Sample: All 138 ASD
vs 124 TD/TIE (excludes training samples)

| Real\Predicted | TD/TIE | ASD |
|---|---|---|
| TD/TIE | 61 | 63 |
| ASD | 38 | 100 |

| | |
|---|---|
| Sensitivity (TP/(TP + FN)): | 0.7246 |
| Specificity (TN/(FP + TN)): | 0.4919 |
| Positive Predictive Value (TP/(TP + FP)): | 0.6135 |
| Negative Predictive Value (TN/(FN + TN)): | 0.6162 |
| Area Under Curve (((TP/(TP + FN)) + (TN/FP + TN))*0.5): | 0.6083 |

According to conventions advocated by Hanley and McNeil (Hanley and McNeil, “The Meaning and Use of the Area Under a Receiver Operating Characteristic (ROC) Curve,” Radiology 143(1):29-36 (1982), which is hereby incorporated by reference in its entirety, diagnostic accuracy may be considered outstanding, excellent, good, fair, or poor if AUC is in the range of 0.9-1.0, 0.81-0.90, 0.71-0.80, 0.61-0.70, or <0.60, respectively.

### Example 6

### Biomarker Panel 6

Among all children, a biomarker panel is described that can differentiate children who go on to develop an ASD, DD, or LD from children who go on to develop typically. This biomarker panel addresses the clinical question: “Is my child more likely to develop an ASD, DD, or LD or to develop typically?”

The optimal model for this classification problem is determined to comprise the expression levels of 31 genetic transcripts in a support vector machine of radial basis function with a cost of 801 and a gamma of 0.01. Expression levels of the genes in this classifier are measured in each individual, and these values are then combined in an equation (which was determined mathematically using a support vector machine-learning algorithm) that yields a dichotomous outcome (a 0 or 1) indicating whether the tested individual is more likely to have the disorder of interest or not. The methods of constructing and deploying support vector machine-learning algorithms on gene-expression data are described in detail by Byvatov and Schneider, “Support Vector Machine Applications in Bioinformatics,” Appl Bioinformatics 2(2):67-77 (2003), which is hereby incorporated by reference in its entirety. The 31 transcripts and their fold-change in ASD relative to typical development are as follows:

| | Gene | Fold-Change |
|---|---|---|
| Illumina Probe | Symbol | (ASD/DD/LD vs. TD/TIE) |
| ILMN_1652662 | GRB10 | 1.05 |
| ILMN_1653468 | ANXA8L1 | 1.04 |
| ILMN_1655403 | ERI2 | −1.06 |
| ILMN_1671627 | AK098672 | 1.04 |
| ILMN_1672966 | CRCP | −1.07 |
| ILMN_1677429 | TWIST2 | −1.04 |
| ILMN_1681757 | RIMKLB | 1.07 |
| ILMN_1688220 | AM393854 | −1.04 |
| ILMN_1689852 | PAQR6 | 1.06 |
| ILMN_1691578 | GTF3C6 | −1.15 |
| ILMN_1705210 | GRASP | 1.07 |
| ILMN_1716279 | CENPE | −1.17 |
| ILMN_1721630 | P2RY4 | −1.05 |
| ILMN_1726701 | BC038536 | −1.04 |
| ILMN_1750457 | ZNF268 | −1.05 |
| ILMN_1757370 | SMPD1 | 1.1 |
| ILMN_1772058 | PANK2 | −1.04 |
| ILMN_1774312 | MRP63 | −1.07 |
| ILMN_1808779 | CSTF3 | −1.1 |
| ILMN_1810228 | TTF2 | −1.11 |
| ILMN_1838667 | AW004814 | 1.06 |
| ILMN_1849670 | AW119108 | 1.04 |
| ILMN_1887013 | AW182429 | −1.04 |
| ILMN_1888639 | Hs.566857 | 1.05 |
| ILMN_1897142 | BX109554 | 1.04 |
| ILMN_2048700 | ATAD2 | −1.16 |
| ILMN_2212878 | ESM1 | −1.08 |
| ILMN_2219466 | APOBEC3B | −1.19 |
| ILMN_2364529 | EZH2 | −1.12 |
| ILMN_2370882 | ACSL5 | −1.1 |
| ILMN_2371055 | EFNA1 | 1.12 |

The table below shows the historical performance of the optimal SVM classifier as determined in the Training Sample after 6-Fold Cross-Validation in 52 individuals with ASD/DD/LD vs 52 TD/TIE individuals. The first lines of the table below, as well as the following tables for Biomarker Panel 6, show the cross-tabulation of the number of individuals who were truly ASD/DD/LD and were called such by the SVM classifier (true-positive or TP calls), the number of individuals who were truly TD/TIE and were called such by the classifier (true-negative or TN calls), the number of individuals who were truly ASD/DD/LD but were called TD/TIE (false-negative or FN calls), and the number of individuals who were truly TD/TIE but were called ASD/DD/LD (false-positive or FP calls). The subsequent lines of the table show the values of sensitivity (the percent of time the model finds true cases), specificity (the percent of time the model avoids calling unaffected individuals cases), positive predictive value (the percent of time those individuals called cases are actually cases) and negative predictive value (the percent of time those individuals called unaffected are actually unaffected), as well as the formulae used for their derivation based on TP, TN, FP, and FN. The final row of the table shows the area under the receiver operating characteristic curve, which is a measure of balance between sensitivity and specificity and can be interpreted as a measure of the model's accuracy.

Performance in Training Sample: 6-Fold Cross-Validation
in 52 ASD/DD/LD vs 52 TD/TIE

| Real/Predicted | TD/TIE | ASD/DD/LD |
|---|---|---|
| TD/TIE | 45 | 7 |
| ASD/DD/LD | 7 | 45 |

| | |
|---|---|
| Sensitivity (TP/(TP + FN)): | 0.8654 |
| Specificity (TN/(FP + TN)): | 0.8654 |
| Positive Predictive Value (TP/(TP + FP)): | 0.8654 |
| Negative Predictive Value (TN/(FN + TN)): | 0.8654 |
| Area Under Curve (((TP/(TP + FN)) + | 0.8654 |
| (TN/FP + TN))*0.5): | |

The tables below show the results of test samples using Biomarker Panel 6 to differentiate between ASD/LD/DD and typical development in all children.

Performance in Test Sample: 26 ASD/DD/LD vs 26 TD/TIE,
12-24 mo. and individually matched on
sex and age within 1 mo. (excludes training samples)

| Real\Predicted | TD/TIE | ASD/DD/LD |
|---|---|---|
| TD/TIE | 23 | 3 |
| ASD/DD/LD | 2 | 24 |

| | |
|---|---|
| Sensitivity (TP/(TP + FN)): | 0.9231 |
| Specificity (TN/(FP + TN)): | 0.8846 |
| Positive Predictive Value (TP/(TP + FP)): | 0.8889 |
| Negative Predictive Value (TN/(FN + TN)): | 0.9200 |
| Area Under Curve (((TP/(TP + FN)) + | 0.9038 |
| (TN/FP + TN))*0.5): | |

Performance in Test Sample: All 54 ASD/DD/LD vs
58 TD/TIE, 12-24 mo. (excludes training samples)

| Real/Predicted | TD/TIE | ASD/DD/LD |
|---|---|---|
| TD/TIE | 49 | 19 |
| ASD/DD/LD | 16 | 38 |

| | |
|---|---|
| Sensitivity (TP/(TP + FN)): | 0.7037 |
| Specificity (TN/(FP + TN)): | 0.7206 |
| Positive Predictive Value (TP/(TP + FP)): | 9.6667 |
| Negative Predictive Value (TN/(FN + TN)): | 9.7538 |
| Area Under Curve (((TP/(TP + FN)) + | 9.7121 |
| (TN/FP + TN))*0.5): | |

Performance in Test Sample: All 172 ASD/DD/LD vs
117 TD/TIE (excludes training samples)

| Real\Predicted | TD/TIE | ASD/DD/LD |
|---|---|---|
| TD/TIE | 69 | 38 |
| ASD/DD/LD | 71 | 101 |

| | |
|---|---|
| Sensitivity (TP/(TP + FN)): | 0.5872 |
| Specificity (TN/(FP + TN)): | 0.6449 |
| Positive Predictive Value (TP/(TP + FP)): | 0.7266 |
| Negative Predictive Value (TN/(FN + TN)): | 0.4929 |
| Area Under Curve (((TP/(TP + FN)) + | 0.6160 |
| (TN/FP + TN))*0.5): | |

According to conventions advocated by Hanley and McNeil (Hanley and McNeil, “The Meaning and Use of the Area Under a Receiver Operating Characteristic (ROC) Curve,” Radiology 143(1):29-36 (1982), which is hereby incorporated by reference in its entirety, diagnostic accuracy may be considered outstanding, excellent, good, fair, or poor if AUC is in the range of 0.9-1.0, 0.81-0.90, 0.71-0.80, 0.61-0.70, or <0.60, respectively.

### Example 7

### Biomarker Panel 7

Among all children, a biomarker panel is described that can differentiate children who go on to develop an ASD from children who go on to develop a DD or LD and from children who go on to develop typically. This biomarker panel addresses the clinical question: “My 12-24 month-old child has developmental red flags. Is he more likely to develop an ASD, to develop a DD or LD, or to develop typically?”

The optimal model for this classification problem is determined to comprise the expression levels of 35 genetic transcripts in a support vector machine of radial basis function with a cost of 601 and a gamma of 0.001. Expression levels of the genes in this classifier are measured in each individual, and these values are then combined in an equation (which was determined mathematically using a support vector machine-learning algorithm) that yields a dichotomous outcome (a 0 or 1) indicating whether the tested individual is more likely to have the disorder of interest or not. The methods of constructing and deploying support vector machine-learning algorithms on gene-expression data are described in detail by Byvatov and Schneider, “Support Vector Machine Applications in Bioinformatics,” Appl Bioinformatics 2(2):67-77 (2003), which is hereby incorporated by reference in its entirety. The 35 transcripts and their fold-change in ASD relative to typical development are as follows:

| | Gene | Fold-Change | Fold-Change | Fold-Change |
|---|---|---|---|---|
| Illumina Probe | Symbol | (ASD vs. TD/TIE) | (DD/LD vs. TD/TIE) | (ASD vs. DD/LD) |
| ILMN_1660403 | GCOM1 | −1.07 | −1.02 | −1.02 |
| ILMN_1665293 | ZBTB20 | 1.05 | 1.07 | 1.07 |
| ILMN_1666918 | LOC729973 | −1.05 | −1.01 | −1.01 |
| ILMN_1668909 | MAP2K3 | −1.06 | −1.03 | −1.03 |
| ILMN_1679113 | BF701780 | 1.04 | 1.06 | 1.06 |
| ILMN_1681757 | RIMKLB | 1.09 | 1.1 | 1.1 |
| ILMN_1681916 | KCNA6 | −1.07 | 1.01 | 1.01 |
| ILMN_1686250 | OR1J4 | 1.05 | 1.05 | 1.05 |
| ILMN_1688676 | SKP1 | 1.06 | 1.01 | 1.01 |
| ILMN_1690105 | STAT1 | −1.55 | −1.17 | −1.17 |
| ILMN_1691364 | STAT1 | −1.46 | −1.16 | −1.16 |
| ILMN_1692834 | C1orf26 | −1.02 | 1.06 | 1.06 |
| ILMN_1700690 | VAT1 | 1.13 | 1.19 | 1.19 |
| ILMN_1723855 | LOC390427 | 1.01 | 1.06 | 1.06 |
| ILMN_1733157 | THSD1 | −1.06 | −1.01 | −1.01 |
| ILMN_1740903 | C7orf49 | 1.1 | 1.06 | 1.06 |
| ILMN_1744962 | SSX5 | 1.06 | 1 | 1 |
| ILMN_1748675 | TMPRSS11B | 1.07 | 1.03 | 1.03 |
| ILMN_1755589 | DIP2B | −1.17 | −1.1 | −1.1 |
| ILMN_1756102 | RFX3 | −1.06 | −1.02 | −1.02 |
| ILMN_1760339 | ZNF774 | 1.08 | 1.05 | 1.05 |
| ILMN_1766204 | GPHA2 | −1.07 | −1.02 | −1.02 |
| ILMN_1768719 | RDH11 | 1.11 | 1.12 | 1.12 |
| ILMN_1771385 | BC050625 | −1.48 | −1.18 | −1.18 |
| ILMN_1778717 | DBF4 | 1.06 | 1.04 | 1.04 |
| ILMN_1779832 | BX248296 | 1.07 | 1.01 | 1.01 |
| ILMN_1803197 | RAB3IP | 1.11 | 1.09 | 1.09 |
| ILMN_1817427 | CD364714 | 1.06 | 1.04 | 1.04 |
| ILMN_1860487 | DA196703 | 1.07 | 1.04 | 1.04 |
| ILMN_1898096 | AA884785 | 1.06 | 1.04 | 1.04 |
| ILMN_1901934 | ZNF33B | 1.15 | 1.14 | 1.14 |
| ILMN_1908878 | AK125234 | 1.06 | 1.02 | 1.02 |
| ILMN_1911278 | AA961268 | −1.06 | −1.01 | −1.01 |
| ILMN_2143427 | LGSN | −1.13 | −1.04 | −1.04 |
| ILMN_2362832 | STAG3L1 | −1.07 | −1.01 | −1.01 |

The table below shows the historical performance of the optimal SVM classifier as determined in the Training Sample after 3-Fold Cross-Validation in 26 individuals with ASD vs 26 DD/LD vs 26 TD/TIE individuals. The first lines of the table below, as well as the following tables for Biomarker Panel 7, show the cross-tabulation of the number of individuals who were truly ASD or DD/LD and were called such by the SVM classifier (true-positive or TP calls), the number of individuals who were truly TD/TIE and were called such by the classifier (true-negative or TN calls), the number of individuals who were truly ASD or DD/LD but were called TD/TIE (false-negative or FN calls), and the number of individuals who were truly TD/TIE but were called ASD or DD/LD (false-positive or FP calls). The subsequent lines of the table show the values of sensitivity (the percent of time the model finds true cases), specificity (the percent of time the model avoids calling unaffected individuals cases), positive predictive value (the percent of time those individuals called cases are actually cases) and negative predictive value (the percent of time those individuals called unaffected are actually unaffected), as well as the formulae used for their derivation based on TP, TN, FP, and FN. The final row of the table shows the area under the receiver operating characteristic curve, which is a measure of balance between sensitivity and specificity and can be interpreted as a measure of the model's accuracy.

Performance in Training Sample: 3-Fold Cross-Validation
in 26 ASD vs 26 DD/LD vs 26 TD/TIE

| Real\Predicted | TD/TIE | DD/LD | ASD |
|---|---|---|---|
| TD/TIE | 15 | 9 | 2 |
| DD/LD | 8 | 15 | 3 |
| ASD | 1 | 3 | 22 |

| | |
|---|---|
| Sensitivity (TP/(TP + FN)): | 0.8462 |
| Specificity (TN/(FP + TN)): | 0.9038 |
| PositivePredictiveValue(TP/(TP + FP)): | 0.8148 |
| Negative Predictive Value (TN/(FN + TN)): | 0.9216 |
| Area Under Curve (((TP/(TP + FN)) + | 0.8750 |
| (TN/FP + TN))*0.5): | |

The tables below show the results of test samples using Biomarker Panel 7 to differentiate between ASD, LD/DD and typical development in all children.

Performance in Test Sample: 13 ASD vs 13 DD/LD vs
13 TD/TIE, 12-24 mo. and individually matched
on sex and age within 1 mo. (excl training samples)

| Real\Predicted | TD/TIE | DD/LD | ASD |
|---|---|---|---|
| TD/TIE | 12 | 0 | 1 |
| DD/LD | 1 | 12 | 0 |
| ASD | 1 | 0 | 12 |

| | |
|---|---|
| Sensitivity (TP/(TP + FN)): | 0.9231 |
| Specificity (TN/(FP + TN)): | 0.9615 |
| Positive Predictive Value (TP/(TP + FP)): | 0.9231 |
| Negative Predictive Value (TN/(FN + TN)): | 0.9615 |
| Area Under Curve (((TP/(TP + FN)) + | 0.9423 |
| (TN/FP + TN))*0.5): | |

Performance in Test Sample: All 61 ASD vs 36 DD/LD vs
51 TD/TIE, 12-24 mo. (excludes training samples)

| Real\Predicted | TD/TIE | DD/LD | ASD |
|---|---|---|---|
| TD/TIE | 34 | 8 | 9 |
| DD/LD | 6 | 18 | 12 |
| ASD | 25 | 7 | 29 |

| | |
|---|---|
| Sensitivity (TP/(TP + FN)): | 0.4754 |
| Specificity (TN/(FP + TN)): | 0.7586 |
| Positive Predictive Value (TP/(TP + FP)): | 0.5800 |
| Negative Predictive Value (TN/(FN + TN)): | 0.6735 |
| Area Under Curve (((TP/(TP + FN)) + | 0.6170 |
| (TN/FP + TN))*0.5): | |

Performance in Test Sample: All 163 ASD vs 37 DD/LD vs
105 TD/TIE (excludes training samples)

| Real\Predicted | TD/TIE | DD/LD | ASD |
|---|---|---|---|
| TD/TIE | 69 | 17 | 19 |
| DD/LD | 7 | 14 | 16 |
| ASD | 78 | 31 | 54 |

| | |
|---|---|
| Sensitivity (TP/(TP + FN)): | 0.3313 |
| Specificity (TN/(FP + TN)): | 0.7535 |
| Positive Predictive Value (TP/(TP + FP)): | 0.6067 |
| Negative Predictive Value (TN/(FN + TN)): | 0.4954 |
| Area Under Curve (((TP/(TP + FN)) + | 0.5424 |
| (TN/FP + TN))*0.5): | |

According to conventions advocated by Hanley and McNeil (Hanley and McNeil, “The Meaning and Use of the Area Under a Receiver Operating Characteristic (ROC) Curve,” Radiology 143(1):29-36 (1982), which is hereby incorporated by reference in its entirety, diagnostic accuracy may be considered outstanding, excellent, good, fair, or poor if AUC is in the range of 0.9-1.0, 0.81-0.90, 0.71-0.80, 0.61-0.70, or <0.60, respectively.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A collection of probes or their complements, said collection of probes recognizing biomarkers comprising at least 50% of the biomarkers from one of the following biomarker sets: (1) ZNF329, LOC641518, TAP1, GBP2, RAB3IP, and MYOF biomarkers; (2) MRPS10, ARF3, CLORF85, KCNE1L, BIN2, CACHD1, CYB5R3, FKBP12-EXI, CHM, DUS4L, STX5, AK3, BU580973, TCRA, CR608770, and SPIT biomarkers; (3) SC65, FUNDC2, NDRG2, RPL28, SRP54, LOC643466, ZDHHC11B, NSUN5B, NDRG3, DHRS3, CPEB2, RAB3IP, PPID, FOXP1, and EFNA1 biomarkers; (4) C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, and MAP4K5 biomarkers; (5) CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, and NSUN5 biomarkers; (6) GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, and EFNA1 biomarkers; or (7) GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1 biomarkers.

2-9. (canceled)

10. The collection of claim 1, wherein said collection of probes recognize at least 50% of the following biomarkers: CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, and NSUN5.

11. The collection of claim 10, wherein said collection of probes have at least 50% of the following nucleotide sequences: SEQ ID NOS: 72, 73, 74, 75, 76, 77, 78, 79, 80, 81, 2, 82, 83, 84, 85, 86, 87, 88, 89, 90, 91, 92, 93, 94, 95, 96, 97, 98, 99, 100, 101, 102, 103, 104, 105, and 106.

12.-42. (canceled)

43. A method of diagnosing whether a subject has an autism spectrum disorder, said method comprising:
obtaining a biological sample from a subject potentially having an autism spectrum disorder;
providing one or more probes recognizing at least 50% of the following biomarkers: ZNF329, LOC641518, TAP1, GBP2, RAB3IP, MYOF, MRPS10, ARF3, CLORF85, KCNE1L, BIN2, CACHD1, CYB5R3, FKBP12-EXI, CHM, DUS4L, STX5, AK3, BU580973, TCRA, CR608770, SPI1, SC65, FUNDC2, NDRG2, RPL28, SRP54, LOC643466, ZDHHC11B, NSUN5B, NDRG3, DHRS3, CPEB2, RAB3IP, PPID, FOXP1, EFNA1, C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, MAP4K5, CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, NSUN5, GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, EFNA1, GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1 biomarkers.
contacting the biological sample from the subject with said collection of probes under conditions effective to permit hybridization of said probes to complementary nucleic acid molecules, if present, in the sample;
detecting any hybridization as a result of said contacting; and
identifying whether the subject has an autism spectrum disorder based on said detecting.

44. A method of diagnosing whether a subject has a predisposition for developing an autism spectrum disorder, said method comprising:
obtaining a biological sample from a subject potentially having a predisposition for developing an autism spectrum disorder;
providing one or more probes recognizing at least 50% of the following biomarkers: ZNF329, LOC641518, TAP1, GBP2, RAB3IP, MYOF, MRPS10, ARF3, CLORF85, KCNE1L, BIN2, CACHD1, CYB5R3, FKBP12-EXI, CHM, DUS4L, STX5, AK3, BU580973, TCRA, CR608770, SPI1, SC65, FUNDC2, NDRG2, RPL28, SRP54, LOC643466, ZDHHC11B, NSUN5B, NDRG3, DHRS3, CPEB2, RAB3IP, PPID, FOXP1, EFNA1, C5ORF44, ARHGAP25, CTDSPL2, CKAP2, MAZ, BET1, SRP54, CR617556, RPE, EHHADH, CMAH, ECD, NMD3, SLC10A7, SNX4, NEDD1, GABPA, MAGMAS, UBE2V2, GBP2, C15ORF44, PCGF6, CABIN1, EIF3J, HS.561844, IMPACT, ATAD2, RGL2, CASD1, TMEM185A, ESM1, ADSSL1, ACSL5, C1ORF124, CYB561, MAP4K5, CRIP1, ING1, LILRB1, SPNS3, CDH11, LOC642403, CASP4, TEAD2, KHDRBS3, FHL3, LOC641518, EPPK1, MARCKSL1, FAM44B, VEGFB, LYRM4, AB007962, PPP2R3B, SPINK2, C9ORF123, PANK2, COG2, CRY2, SESN1, EPN2, IL23A, BE439556, DB050967, TMEM203, RCBTB2, ZNF627, CMTM1, HSD11B1L, MAL, TOP1MT, NSUN5, GRB10, ANXA8L1, ERI2, AK098672, CRCP, TWIST2, RIMKLB, AM393854, PAQR6, GTF3C6, GRASP, CENPE, P2RY4, BC038536, ZNF268, SMPD1, PANK2, MRP63, CSTF3, TTF2, AW004814, AW119108, AW182429, HS.566857, BX109554, ATAD2, ESM1, APOBEC3B, EZH2, ACSL5, EFNA1, GCOM1, ZBTB20, LOC729973, MAP2K3, BF701780, RIMKLB, KCNA6, OR1J4, SKP1, STAT1, C1ORF26, VAT1, LOC390427, THSD1, C7ORF49, SSX5, TMPRSS11B, DIP2B, RFX3, ZNF774, GPHA2, RDH11, BC050625, DBF4, BX248296, RAB3IP, CD364714, DA196703, AA884785, ZNF33B, AK125234, AA961268, LGSN, and STAG3L1 biomarkers.
contacting the biological sample from the subject with said collection of probes under conditions effective to permit hybridization of said probes to complementary nucleic acid molecules, if present, in the sample;
detecting any hybridization as a result of said contacting; and
identifying whether the subject has a predisposition for developing an autism spectrum disorder based on said detecting.

45. The method according to claim 43, wherein the subject is between 11 months to 4 years of age.

46. The method according to claim 43, wherein the biological sample is sputum, blood, a blood fraction, tissue or fine needle biopsy sample, urine, stool, peritoneal fluid, or pleural fluid.

47. The method according to claim 46, wherein the biological sample is blood.

48. The method according to claim 43, wherein the biological sample is isolated peripheral blood mononuclear cells.

49. The method according to claim 43, wherein said detecting any hybridization comprises measuring RNA expression level.

50. The method according to claim 49, wherein the RNA expression level is measured using a nucleic acid hybridization assay or a nucleic acid amplification assay.

51. The method according to claim 50, wherein the nucleic acid hybridization assay is carried out using an array comprising the collection of probes.

52. The method according to claim 43, wherein the autism spectrum disorder is selected from the group consisting of autistic disorder and pervasive development disorder.

53. The method according to claim 44, wherein the subject is between 11 months to 4 years of age.

54. The method according to claim 44, wherein the biological sample is sputum, blood, a blood fraction, tissue or fine needle biopsy sample, urine, stool, peritoneal fluid, or pleural fluid.

55. The method according to claim 54, wherein the biological sample is blood.

56. The method according to claim 44, wherein said detecting any hybridization comprises measuring RNA expression level.

57. The method according to claim 56, wherein the RNA expression level is measured using a nucleic acid hybridization assay or a nucleic acid amplification assay.

58. The method according to claim 57, wherein the nucleic acid hybridization assay is carried out using an array comprising the collection of probes.

59. The method according to claim 44, wherein the autism spectrum disorder is selected from the group consisting of autistic disorder and pervasive development disorder.


SEQUENCE LISTING (165 sequences)

SEQ ID NO: 1 - DNA, 50 bp, Artificial
  primer
ccccaccactacctccatggttgttcatttaggatgcttctaattcagcc

SEQ ID NO: 2 - DNA, 50 bp, Artificial
  primer
ggggctatattctcaggcttcctgggctctgttggcattgggcaagcacg

SEQ ID NO: 3 - DNA, 50 bp, Artificial
  primer
gtaacggagtttagagccagggctgatgctttggtgtggccagcactctg

SEQ ID NO: 4 - DNA, 50 bp, Artificial
  primer
gcctgtccagctccctctccccaagaaacaacatgaatgagcaacttcag

SEQ ID NO: 5 - DNA, 50 bp, Artificial
  primer
ggaactctgatgctctgcgtgggaccatgcctgaactccccgaataactg

SEQ ID NO: 6 - DNA, 50 bp, Artificial
  primer
gccatgtcaccgagccccattgattcccagagggtcttagtcctggaaag

SEQ ID NO: 7 - DNA, 50 bp, Artificial
  primer
gtagaagcaggtccatgtcttttgtggtttcctgcacatctttggagtag

SEQ ID NO: 8 - DNA, 50 bp, Artificial
  primer
agggaccaatctggggctggaaatgttaggaggttgccttggtgctgccc

SEQ ID NO: 9 - DNA, 50 bp, Artificial
  primer
gggacagggctattgataaggtccccttggtgttgccttcttgcatctcc

SEQ ID NO: 10 - DNA, 50 bp, Artificial
  primer
tcttgcctccccagttttcatcccagtggtaacgcctgatttttggtagc

SEQ ID NO: 11 - DNA, 50 bp, Artificial
  primer
ggttgcttcagagcctggagaggcaaagaagatggaagacaaggaaaagg

SEQ ID NO: 12 - DNA, 50 bp, Artificial
  primer
cacggttaaaagctgctgccagttagccaagacattatccaccaaattgc

SEQ ID NO: 13 - DNA, 50 bp, Artificial
  primer
gggccctcccagaacctcagcatttccttccagcccatccaaacactgag

SEQ ID NO: 14 - DNA, 50 bp, Artificial
  primer
tccctgggccagcagggacctctgaagccttctttgtggccttatttttt

SEQ ID NO: 15 - DNA, 50 bp, Artificial
  primer
ctgccatagttacctggattgtcagccttggtagcctttgtctaaagtcc

SEQ ID NO: 16 - DNA, 50 bp, Artificial
  primer
gtgcctctcagccgttgattgtaactttaaagtcccatggttttggagtg

SEQ ID NO: 17 - DNA, 50 bp, Artificial
  primer
ggaacaggaggaaaccattcagaggatcgacgagaacgtgctaggagccc

SEQ ID NO: 18 - DNA, 50 bp, Artificial
  primer
gtggctattactttgttcttggtccttcacagggcctgctccatcccacc

SEQ ID NO: 19 - DNA, 50 bp, Artificial
  primer
gcctgttgctttggcttgtattttcagtgctgtgttgaataagagtggtg

SEQ ID NO: 20 - DNA, 50 bp, Artificial
  primer
gcgcgaagagcaactatcagttaatctggggcgctgggaccaagctaatt

SEQ ID NO: 21 - DNA, 50 bp, Artificial
  primer
tgcacactgccctattgtagtcaagtcttacccccacctcacatctcctg

SEQ ID NO: 22 - DNA, 50 bp, Artificial
  primer
acgccagctgggcgtcagaccccaccggggcaaccttgcagaggacgacc

SEQ ID NO: 23 - DNA, 50 bp, Artificial
  primer
tctggaccacaggatggtggtggcattgcaggttggcaagtgggctgatg

SEQ ID NO: 24 - DNA, 50 bp, Artificial
  primer
cctgcttccccacactctgcctctcctgacatctgggtctctgggttatg

SEQ ID NO: 25 - DNA, 50 bp, Artificial
  primer
gttggaatgggagttggcgggcagtgaacgagtgtggggaaggattggtg

SEQ ID NO: 26 - DNA, 50 bp, Artificial
  primer
cccaagcacctggaagacatgccagatccatgtgcagtaatgcctggtgg

SEQ ID NO: 27 - DNA, 50 bp, Artificial
  primer
gttatatagctatacagaaatggatcctgtcatcattgcttctgaaggag

SEQ ID NO: 28 - DNA, 50 bp, Artificial
  primer
ttgactcaaacccacggtgcgcctcgggccgttaggggtaccccgaggca

SEQ ID NO: 29 - DNA, 50 bp, Artificial
  primer
tggggcatgtgggctctgcactacctccagctgaccatggcattcagttg

SEQ ID NO: 30 - DNA, 50 bp, Artificial
  primer
cggggcggggaagtgaaccccgacggtcagcgctttgtcatctggtttca

SEQ ID NO: 31 - DNA, 50 bp, Artificial
  primer
ctactgttaggtgagggagtcacagccagacagagagtattgctggaggg

SEQ ID NO: 32 - DNA, 50 bp, Artificial
  primer
tgacccccacagggaggcaggaaaacagccagaagccaccttgacacttt

SEQ ID NO: 33 - DNA, 50 bp, Artificial
  primer
tcagagcagtggctggggcactggaagtatgtcctggggagcaatgcatg

SEQ ID NO: 34 - DNA, 50 bp, Artificial
  primer
caggtgtgagccaccgtgcccggccaagtaaaatgttttttaaaatggtt

SEQ ID NO: 35 - DNA, 50 bp, Artificial
  primer
agaatattggatgacatttcctgacatgtgggagggagaaactccctaac

SEQ ID NO: 36 - DNA, 50 bp, Artificial
  primer
cctgcctttaagccaaagaaacaagctgtgcaggcatggtcccttaaggc

SEQ ID NO: 37 - DNA, 50 bp, Artificial
  primer
cagggcccacgtgtatagtatctgtatataagttgctgtgtgtctgtcct

SEQ ID NO: 38 - DNA, 50 bp, Artificial
  primer
tgtgggtcaaaagtgccggtcaaaatggaagtgaatccccctaaacagga

SEQ ID NO: 39 - DNA, 50 bp, Artificial
  primer
gggaaagagtgaaaagacaagaagggcgcaaactgtgacagactcaccgc

SEQ ID NO: 40 - DNA, 50 bp, Artificial
  primer
cctgtggcgcagtacactcccaagccaccaatgcagttaatatgctctca

SEQ ID NO: 41 - DNA, 50 bp, Artificial
  primer
cttgcgtcccttggactgcctgttgattgatggaaagtgtctgcactgac

SEQ ID NO: 42 - DNA, 50 bp, Artificial
  primer
gttggttgcgggggagaggggagaatggagtagagtcccttggtacaagc

SEQ ID NO: 43 - DNA, 50 bp, Artificial
  primer
ggaaacttagtgggagagtaacagaatgcctggagagcctgactctgagc

SEQ ID NO: 44 - DNA, 50 bp, Artificial
  primer
cagcatagacttaactcccttaagcccagacatctgttgggacctgaccc

SEQ ID NO: 45 - DNA, 50 bp, Artificial
  primer
tttggaggtaatattgggttgaattctgactgcccctttctagctggacc

SEQ ID NO: 46 - DNA, 50 bp, Artificial
  primer
ccagcacagggaacttaggttagtgtggcaagcctttcctcttctggtct

SEQ ID NO: 47 - DNA, 50 bp, Artificial
  primer
caccacccaactggaagtcattcctgatgtgctgtgagcagaatgggcct

SEQ ID NO: 48 - DNA, 50 bp, Artificial
  primer
gccccaaatgtggccctcttggcacaagtctttgtgatctcagctgtggg

SEQ ID NO: 49 - DNA, 50 bp, Artificial
  primer
ggtagggaaattagggttcagtttatcactggacattcaggaggcaagtc

SEQ ID NO: 50 - DNA, 50 bp, Artificial
  primer
ctacgagaacacttttctgtgtttcccccatgccgtcctgtcacatcctc

SEQ ID NO: 51 - DNA, 50 bp, Artificial
  primer
ctgcttggctctctacacatggcatttcagggtataagatgtagcatttc

SEQ ID NO: 52 - DNA, 50 bp, Artificial
  primer
ctgtcactgctggagttgccagttcactctcagaaaaaatagccgacagc

SEQ ID NO: 53 - DNA, 50 bp, Artificial
  primer
ggttttgcaccatcctcttacggcctagagagttgacaagttgcttgtag

SEQ ID NO: 54 - DNA, 50 bp, Artificial
  primer
ttctacctgcagtcaaaggtggtccgcgcaaaggagcgcctggatgagga

SEQ ID NO: 55 - DNA, 50 bp, Artificial
  primer
caagatgaaagcgtgtggagaagtgtcagatggcagtggaagcatgtgtg

SEQ ID NO: 56 - DNA, 50 bp, Artificial
  primer
gtgactgtctggatcaaacccagcggcctgcagacagatgtacagaagat

SEQ ID NO: 57 - DNA, 50 bp, Artificial
  primer
gaggtctagaagtacctaaacctgctgttccacagccagtcccttcaagc

SEQ ID NO: 58 - DNA, 50 bp, Artificial
  primer
tgactttgtaaatctgcccacacccagctggccatatccacccctcgacg

SEQ ID NO: 59 - DNA, 50 bp, Artificial
  primer
gccaggtccttatgttgtcaccatagagcaacaaaggtatagggctgcct

SEQ ID NO: 60 - DNA, 50 bp, Artificial
  primer
caaccttagttcacagtgcagtaccacccctcaccttccatgacttgtcc

SEQ ID NO: 61 - DNA, 50 bp, Artificial
  primer
ggctcgtcttgagctcctggcctcaatcgatcttcctgccaaggttttgg

SEQ ID NO: 62 - DNA, 50 bp, Artificial
  primer
ggctttggcaattctttctcagcctacaccctcacttgttgtggatcatg

SEQ ID NO: 63 - DNA, 50 bp, Artificial
  primer
cagaaattcagaaagggagccagccaccctggggcagtgaagtgccactg

SEQ ID NO: 64 - DNA, 50 bp, Artificial
  primer
gccagtatcacatatggctggcagcggacacaaggggtatcttggtactg

SEQ ID NO: 65 - DNA, 50 bp, Artificial
  primer
acgtgtgcgtgccgtttctccaagcactgcaggttccaccgtgtgtcaga

SEQ ID NO: 66 - DNA, 50 bp, Artificial
  primer
ctgctgatgtagttcccgggttacctgtatctgaaggacggttctggggc

SEQ ID NO: 67 - DNA, 50 bp, Artificial
  primer
agggcaaaggcaaggtggtggacctgctggccacggacgccgacatcatc

SEQ ID NO: 68 - DNA, 50 bp, Artificial
  primer
ttagtaaccacaagttcaagggtcaaagggaccctctgtgccttcttctt

SEQ ID NO: 69 - DNA, 50 bp, Artificial
  primer
gccatcccaggatgtgagtgggtctgaagatacattcccaaataaacgac

SEQ ID NO: 70 - DNA, 50 bp, Artificial
  primer
ggggccagtctcctctaatgctcagatttcccatagttggcttttgctgt

SEQ ID NO: 71 - DNA, 50 bp, Artificial
  primer
gggttgtcgttttggaaagtaggccaacagaaaatcctactgcacacagc

SEQ ID NO: 72 - DNA, 50 bp, Artificial
  primer
gccgagagccacactttcaagtaaaccaggtggtggagaccccatccttg

SEQ ID NO: 73 - DNA, 50 bp, Artificial
  primer
gcttgggtacacttctcttaagtggtctagtcaaggaacctcaagtcatg

SEQ ID NO: 74 - DNA, 50 bp, Artificial
  primer
tgtcagatgcgtctctgctgacctgagtctgccctgcaccatggacctgc

SEQ ID NO: 75 - DNA, 50 bp, Artificial
  primer
gcacatctgccacttttgaattcccggctggagagctggcaggaccctgt

SEQ ID NO: 76 - DNA, 50 bp, Artificial
  primer
cgtgccagatataactgtcttgtttcagtgagagacgccctatttctatg

SEQ ID NO: 77 - DNA, 50 bp, Artificial
  primer
aatggtatggacagtaaggtgcaggctgaggaggtctcagatggagatga

SEQ ID NO: 78 - DNA, 50 bp, Artificial
  primer
actccaagggccaaagctcaaatgcccaccatagaacgactgtccatgac

SEQ ID NO: 79 - DNA, 50 bp, Artificial
  primer
agtgggtgctagggtcttgactttatctccgctgcacaagcagtgtgttg

SEQ ID NO: 80 - DNA, 50 bp, Artificial
  primer
aggcaccttcagcgaggacagcaaagggcgtctacagagaccagccatat

SEQ ID NO: 81 - DNA, 50 bp, Artificial
  primer
aggtctcctatgggtgcctgggaagtccttgaaagtggactgttctcagg

SEQ ID NO: 82 - DNA, 50 bp, Artificial
  primer
ccagtgtctactatttagtgccctggctctatttcggtcctcctccccgg

SEQ ID NO: 83 - DNA, 50 bp, Artificial
  primer
cctgagccagaagtggggtgcttatactcccaaaccttgagtgtccagcc

SEQ ID NO: 84 - DNA, 50 bp, Artificial
  primer
taggcataaggaaactcgtttgcaggctctctgtccagggctgcttcctg

SEQ ID NO: 85 - DNA, 50 bp, Artificial
  primer
ggacagagttggaagaggagactgggaggcagcaagaggggtcacatacc

SEQ ID NO: 86 - DNA, 50 bp, Artificial
  primer
accggtgcccaaatctggctggtggacagaagcacctggagagttggaga

SEQ ID NO: 87 - DNA, 50 bp, Artificial
  primer
cttatcagaacggagtggatggtcaccttcctcacttattgctaatctcc

SEQ ID NO: 88 - DNA, 50 bp, Artificial
  primer
cgttcctgtgaagctctccctgacatgcatcttcgtctctccatcctggc

SEQ ID NO: 89 - DNA, 50 bp, Artificial
  primer
gctcctggcagtcaccttcgcagcctctctgatccctcaatttggtctgt

SEQ ID NO: 90 - DNA, 50 bp, Artificial
  primer
cgctgagtagtctgaggagcaagaggagttggtctttctgtcccagtggc

SEQ ID NO: 91 - DNA, 50 bp, Artificial
  primer
gctggcggcctcgacggcagctgcggaactaggccgagggacaaaggcta

SEQ ID NO: 92 - DNA, 50 bp, Artificial
  primer
gagaactcctgggctttctaaagaggctgcgggaagccatcctccactcc

SEQ ID NO: 93 - DNA, 50 bp, Artificial
  primer
tgacccaagggccagcatggggaagagatggttgcaggcaaaatgcactt

SEQ ID NO: 94 - DNA, 50 bp, Artificial
  primer
cctgacactggagggcagctgtcttgtgcattacttgtgttcccagcacc

SEQ ID NO: 95 - DNA, 50 bp, Artificial
  primer
actgatcatgaagccaccggccactgccacgcatgttgcacctgtgccat

SEQ ID NO: 96 - DNA, 50 bp, Artificial
  primer
ggagcttctaacccgtcatggttcaatacacattcttcttttgccagcgc

SEQ ID NO: 97 - DNA, 50 bp, Artificial
  primer
ccctctccctggggcatgtgcagagtttcaacttcttgttggctgctctc

SEQ ID NO: 98 - DNA, 50 bp, Artificial
  primer
caggacactctccaaactctccctccagtctaagatgatgccagctccag

SEQ ID NO: 99 - DNA, 50 bp, Artificial
  primer
gaactctgctgtccaggcactgcttggcttactatcccagcaaagactgc

SEQ ID NO: 100 - DNA, 50 bp, Artificial
  primer
tcacataaggtcctttgcttttctttgtgttaagagggacttgcctctgt

SEQ ID NO: 101 - DNA, 50 bp, Artificial
  primer
gtgctctcttgctaaagaccgcagagaccatacctgttgtcaaagagggt

SEQ ID NO: 102 - DNA, 50 bp, Artificial
  primer
tgtacccaaggcacagcgcaacatctcagcgaagaccgcaccccggaagc

SEQ ID NO: 103 - DNA, 50 bp, Artificial
  primer
ggggacttgcaaggcctcacctgtttggccatgattgatgacgtgactgc

SEQ ID NO: 104 - DNA, 50 bp, Artificial
  primer
acttgagctgaaaacccagatggtgttaactggccgccccactttccggc

SEQ ID NO: 105 - DNA, 50 bp, Artificial
  primer
caggatcagcattgcctggtgcaagcggttcagggtgccagtggagaaga

SEQ ID NO: 106 - DNA, 50 bp, Artificial
  primer
acgtgctccctctgccaggaggagaatgaagacgtggtgcgagatgcgct

SEQ ID NO: 107 - DNA, 50 bp, Artificial
  primer
gcgctcggagacccggtggagcccaaagtttccgcgcagcccctgggtgg

SEQ ID NO: 108 - DNA, 50 bp, Artificial
  primer
ggctgtggccaacactacgtgagcccctcccttcccagtgggcaccatgt

SEQ ID NO: 109 - DNA, 50 bp, Artificial
  primer
ttactgccagcatctcagcctgaggaaaacgtagactgtacagttcccat

SEQ ID NO: 110 - DNA, 50 bp, Artificial
  primer
gcagaaagttgaggcttagaaggtgcacggccctacctcttccgaggtgc

SEQ ID NO: 111 - DNA, 50 bp, Artificial
  primer
gaaagatttcctccacggcctttgccccagttgtggggaggtctctgtgc

SEQ ID NO: 112 - DNA, 50 bp, Artificial
  primer
gcagagcgacgagatggacaataagatgaccagctgcagctacgtggccc

SEQ ID NO: 113 - DNA, 50 bp, Artificial
  primer
gtaaatgcaccggtttggattcaggcacagccccagtctgcctacagcag

SEQ ID NO: 114 - DNA, 50 bp, Artificial
  primer
tccagtagctgattacaactacaacccacacccaaggggatggagacgcc

SEQ ID NO: 115 - DNA, 50 bp, Artificial
  primer
gccttgagctcagagggggtacccaggcgggcagggaccgtccaggccca

SEQ ID NO: 116 - DNA, 50 bp, Artificial
  primer
cggagaagacgaggaagaggaggagcagttggttctggtggaattatcag

SEQ ID NO: 117 - DNA, 50 bp, Artificial
  primer
tcccatgaagccctctcctcagctttacttgctcccccgcccttagcctt

SEQ ID NO: 118 - DNA, 50 bp, Artificial
  primer
ccagaggtgcaaaatgcaggagcagagagtgtggattctcagccaggtcc

SEQ ID NO: 119 - DNA, 50 bp, Artificial
  primer
cttgctcactggggacaaatatcgacgtcagctccgtcagctctgtggtg

SEQ ID NO: 120 - DNA, 50 bp, Artificial
  primer
gggctaacacattaatgagatttcctgagactgccctgtagagatgcttg

SEQ ID NO: 121 - DNA, 50 bp, Artificial
  primer
gtttggtgtctggtgagggcctgctctgtgcttccaagatgacgccttgt

SEQ ID NO: 122 - DNA, 50 bp, Artificial
  primer
ctcctttcctggagctggtttagctggatatgggagggggtttggctgcc

SEQ ID NO: 123 - DNA, 50 bp, Artificial
  primer
gtcaaagttgtatggctgaggcccacacggtggctcacttctgtaatccc

SEQ ID NO: 124 - DNA, 50 bp, Artificial
  primer
gtggcagaaccacattttgttccctcttcaagggtgtcttgtatgtgccg

SEQ ID NO: 125 - DNA, 50 bp, Artificial
  primer
cagccatctctgcagttctctcagtgcaggcagttcttcctctcaggctg

SEQ ID NO: 126 - DNA, 50 bp, Artificial
  primer
aaagaatgacccctgttgatgagaggtgttacatgtggccatgggacagg

SEQ ID NO: 127 - DNA, 50 bp, Artificial
  primer
cacggtctggaaggtggaaagtccaagatcatggctctggcaggtctctg

SEQ ID NO: 128 - DNA, 50 bp, Artificial
  primer
ggcttcagagctgtatttcggccaacccagtctttgttcagtttctagcg

SEQ ID NO: 129 - DNA, 50 bp, Artificial
  primer
ttgcagggggcagacagatttattatgaggaattgactcctgtgattatg

SEQ ID NO: 130 - DNA, 50 bp, Artificial
  primer
gcctgtgagatgggttgcagaattgacctgagcagaccaggcttccgagg

SEQ ID NO: 131 - DNA, 50 bp, Artificial
  primer
accctttggtccttcgacggcgccagacctacttgtgctatgaggtggag

SEQ ID NO: 132 - DNA, 50 bp, Artificial
  primer
agtgtgaccctgacctctgtcttacttgtggagccgctgaccattgggac

SEQ ID NO: 133 - DNA, 50 bp, Artificial
  primer
attgcagaatgtgaagaagttagaagaaaaagtgaactgtttaaccctgt

SEQ ID NO: 134 - DNA, 50 bp, Artificial
  primer
tgttttcttaacttcctaacctatggttaattattctgaccagtggaaag

SEQ ID NO: 135 - DNA, 50 bp, Artificial
  primer
gaggagccatagcagcaaggagtacctggagctgcacagggagaacttcc

SEQ ID NO: 136 - DNA, 50 bp, Artificial
  primer
gggacctttggagcacagcctacgatcctggtgcaaggccggtggatgca

SEQ ID NO: 137 - DNA, 50 bp, Artificial
  primer
agcactgtcgaggtcacagactgcagacctccttgtatcttcaaatggag

SEQ ID NO: 138 - DNA, 50 bp, Artificial
  primer
cccctccctacctcatggggaatgtctgggaagctggggacattgctatg

SEQ ID NO: 139 - DNA, 50 bp, Artificial
  primer
ggccacattggggtcaccatcctcaaggctccatctactaagggcatctt

SEQ ID NO: 140 - DNA, 50 bp, Artificial
  primer
ttgaagttgatgtggaaattgccaaacaatctgtgactctcaaaatcatg

SEQ ID NO: 141 - DNA, 50 bp, Artificial
  primer
tactccaggccaaaggaagcaccagagccaatggaacttgatggccctaa

SEQ ID NO: 142 - DNA, 50 bp, Artificial
  primer
cgccatcacagctgaacttgttgagatccccgtgttactgcctatcagca

SEQ ID NO: 143 - DNA, 50 bp, Artificial
  primer
gggcttgtcacttttcagtacatttctagtggggaagagcagggcgaccc

SEQ ID NO: 144 - DNA, 50 bp, Artificial
  primer
aggacctgggccattgcaaccaaaatggggacttcctgggtagggaggtc

SEQ ID NO: 145 - DNA, 50 bp, Artificial
  primer
ccacgtttctgtgacttgagtgccctgggagttttacgctgcctcgtctc

SEQ ID NO: 146 - DNA, 50 bp, Artificial
  primer
gagcatttccaagaggcaagtggaacccgtggtccattaaaccctctccc

SEQ ID NO: 147 - DNA, 50 bp, Artificial
  primer
gcaagagggacaggagcccagaagagacactgaggacaagagatcacacc

SEQ ID NO: 148 - DNA, 50 bp, Artificial
  primer
ctctgccttcccaacaagggtctgtacatctttcagggacagactgctcc

SEQ ID NO: 149 - DNA, 50 bp, Artificial
  primer
gaggtcatcaagggcagagaactgtgtggcatctccttatagtaaaagtg

SEQ ID NO: 150 - DNA, 50 bp, Artificial
  primer
caggctgtttagggaccattgcctgtcttggtcacatgagtctgtctcct

SEQ ID NO: 151 - DNA, 50 bp, Artificial
  primer
ggcgtgcaaccaagcctcctgaatccaattcacagcgagcacattgtcac

SEQ ID NO: 152 - DNA, 50 bp, Artificial
  primer
tgctactgtcttcaagcaccccaaatagagaaaacctgggcgtcagtggc

SEQ ID NO: 153 - DNA, 50 bp, Artificial
  primer
aaagtacagctgcagtgtgtggggagccggagggaggagctcgagatctt

SEQ ID NO: 154 - DNA, 50 bp, Artificial
  primer
ggaggaattgagggcaagcacccaggactgatgaggtcttaacaaaaacc

SEQ ID NO: 155 - DNA, 50 bp, Artificial
  primer
ccatgggccttttcacaggggacacaggcttcttaaaacaacccggcttc

SEQ ID NO: 156 - DNA, 50 bp, Artificial
  primer
gactctaccccctaatttggtaggagatgaaggagaaaaggatggcattg

SEQ ID NO: 157 - DNA, 50 bp, Artificial
  primer
ctgctacttgaccctagagctctttgactccgattcccatgtacgtgccc

SEQ ID NO: 158 - DNA, 50 bp, Artificial
  primer
gcaaggctggggtgctttgagattgtccatgtctgatctacgcaccacgc

SEQ ID NO: 159 - DNA, 50 bp, Artificial
  primer
gggggagggagcgtgaagaattgtttaacatttggccatcgtttggctgc

SEQ ID NO: 160 - DNA, 50 bp, Artificial
  primer
ccctcagagtcagtgatccaagagtgtaagctaccagtgcttcctgggac

SEQ ID NO: 161 - DNA, 50 bp, Artificial
  primer
gctggaaatgtgggggcaacaaatacattagtgaacaccctggcggatcc

SEQ ID NO: 162 - DNA, 50 bp, Artificial
  primer
gaggccctggaggatggcagagggaacaggaggaaaacaaagtcacatgc

SEQ ID NO: 163 - DNA, 50 bp, Artificial
  primer
accaccagctttcctggttctccaccttgcagaaagaagatcctgggact

SEQ ID NO: 164 - DNA, 50 bp, Artificial
  primer
gcaaacttaccagatcttgtcagtcatttcctatgtgtatgttgacctgg

SEQ ID NO: 165 - DNA, 50 bp, Artificial
  primer
tggccaggatggtcttgatctcttgaccttgtgatccacctgcctcatca